(19) United States
(12) Patent Application Publication
(10) Pub. No.: US 2011/0256628 A1
(43) Pub. Date: Oct. 20, 2011

(54) ADAPTIVE TISSUE ENGINEERING SCAFFOLD

(75) Inventors: Anna Galperin, Seattle, WA (US); Thomas Joseph Long, Seattle, WA (US); Buddy D. Ratner, Seattle, WA (US)

(73) Assignee: The University of Washington through its Center for Commercialization, Seattle, WA (US)

(21) Appl. No.: 13/090,999

(22) Filed: Apr. 20, 2011

Related U.S. Application Data
(60) Provisional application No. 61/326,055, filed on Apr. 20, 2010.

(51) Int. Cl. C12N 5/071; C12N 5/0735; C12N 5/0775; C12N 5/0786; C08J 9/00; C08J 9/26

(52) U.S. Cl. 435/396; 521/149; 521/61; 521/50.5

(57) ABSTRACT

The embodiments described herein include porous scaffolds formed from a stimuli-responsive polymer. The stimuli-responsive polymer of the scaffold creates a “smart” scaffold that changes properties in response to an effective stimulus applied to the stimuli-responsive polymer. In a preferred embodiment, an effective stimulus applied to the scaffold initiates a phase transition event in the stimuli-responsive polymer that results in a change in the volume of the pores of the scaffold. The scaffolds can be used to capture appropriately sized objects (e.g., cells) by using the volume-change properties of the pores. Relatedly, the scaffolds can be used as tissue-engineering scaffolds by capturing cells in the pores and introducing the cell-loaded scaffold into a cell-growth environment (e.g., in vivo).

### CROSS-REFERENCE TO RELATED APPLICATION

This application claims the benefit of U.S. Provisional Application No. 61/326,055, filed Apr. 20, 2010, the disclosure of which is incorporated herein by reference in its entirety.

### STATEMENT OF GOVERNMENT LICENSE RIGHTS

This invention was made with Government support under grant No. R01 HL64387 awarded by the National Institutes of Health. The Government has certain rights in the invention.

### BACKGROUND

Hydrogels are networks of hydrophilic cross-linked polymers that swell to equilibrium in the presence of water or physiological fluids. During the last two decades, naturally derived and synthetic hydrogels have been topics of extensive research in the field of tissue engineering for application as three-dimensional porous scaffolds for the repair and regeneration of various tissues and organs. Hydrogels are appealing for this goal due to their water content and mechanical properties that mimic those of living tissues. The advantages of synthetic hydrogels compared to natural ones are reproducibility, freedom from biological contamination, and the ability to tune properties by synthetic chemistry modifications.

So called “intelligent hydrogels” or “smart hydrogels” have been a subject of special interest due to their ability to respond with pronounced property changes to external stimuli. For example, thermosensitive degradable hydrogels have been used for cell entrapment and protein release. Among the intelligent hydrogels, thermosensitive poly(N-isopropyl acrylamide) (polyNIPAM) is probably one of the most extensively studied. In aqueous media polyNIPAM hydrogels exhibit a volume phase transition temperature (VPTT) at around 32-34° C. The cross-linked polyNIPAM network undergoes an abrupt, reversible swelling-deswelling process below and above the VPTT. The VPTT of polyNIPAM hydrogels can be modulated by copolymerization with hydrophilic or hydrophobic monomers to increase or decrease the transition temperature. Due to their thermosensitive nature, biostability, and biocompatibility, polyNIPAM-based hydrogels are attractive candidates for biomedical applications including tissue engineering.

Three-dimensional polymer scaffolds that mimic native extracellular matrices play a pivotal role in tissue engineering. The function of scaffolds is to direct growth of cells, either seeded within the porous structure or migrating from the surrounding tissue. Therefore, scaffold matrices must provide cell adhesion, proliferation, differentiation, and infiltration. One of the criteria for the successful application of three dimension scaffolds is high porosity. Various techniques have been used to fabricate interconnected porous scaffolds such as salt leaching, phase separation methods, gas foaming, fiber meshes, and electrospinning. However, these methods do not provide precision control of pore size, structure, or interconnectivity. The criteria for optimal porous scaffold biointegration and angiogenesis requiring that all pores are identical in size, well-interconnected, and between 30 and 40 μm in diameter. Degradability is an additional important factor in designing scaffolds for applications in tissue and organ regeneration. Ideally, the scaffold should provide mechanical and biochemical support during the regeneration process and then, as new tissue forms, should be completely degraded and eliminated through metabolic processes.

There are examples in the scientific literature of partially degradable polyNIPAM hydrogels where the degradable bonds are typically introduced into the cross-linking sites but not within the polymer backbone. Different biodegradable crosslinkers have been used for this purpose, such as amino-acid derivatives, degradable polyaspartic acid derivatives, as well as modified dextran, polylactic acid, and poly(ε-caprolactone) (PCL). In all these cases, materials degrade through cleavage of cross-linkings leaving a nondegradable, high molecular weight polyNIPAM backbone. Such polyNIPAM chains have limited ability to be eliminated from the body by natural pathways and may induce the foreign body response.

Despite recent advances in biocompatible scaffolds, improvements in scaffold functionality and materials are desirable for the next generation of scaffolds to be realized.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a scaffold is provided. In one embodiment, the scaffold is formed from a stimuli-responsive polymer, the scaffold having a plurality of interconnected pores that each has a volume that changes in relation to a phase transition event, wherein the phase transition event is initiated by an effective stimulus to the stimuli-responsive polymer.

In another aspect, a method is provided for forming a scaffold as provided herein. In one embodiment, the method comprises the steps of:

(a) providing a template comprising a plurality of packed particles in a vessel, said packed particles being packed in an arrangement such that voids in between the particles can be infiltrated by a liquid poured into the vessel;

(b) filling the voids in between the particles with a solution comprising a monomer for the stimuli-responsive polymer and a polymerization initiator;

(c) polymerizing the monomer for the stimuli-responsive polymer using the polymerization initiator to provide the stimuli-responsive polymer as a matrix surrounding the packed particle template, thereby providing a particle-scaffold composite; and

(d) dissolving the packed particles from the particle-scaffold composite to provide the scaffold.

In another aspect, a method is provided for growing cells using a scaffold as provided herein. In one embodiment, the scaffold has a large pore state and a small pore state depending on whether a volume phase transition event has occurred, the method comprising the steps of:

(a) contacting a suspension of cells with the scaffold in the large pore state such that the cells infiltrate the large pores of the scaffold;

(b) applying an effective stimulus to the scaffold so as to transition the scaffold to the small pore state, thereby trapping the cells in the pores;

(c) placing the scaffold containing trapped cells in a location where cell growth is desired;

(d) culturing the cells within the scaffold, which provides mechanical and biochemical support for the cells; and

(e) biodegrading the scaffold to provide space for aggregated cells or newly formed tissue.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a reaction scheme for the synthesis of a fully biodegradable pNIPAM-based hydrogel in accordance with the embodiments provided herein. FIG. 2 graphically illustrates the degradation of poly(NIPAM-TEGDMA), poly(NIPAMTEGDMA)-20 and poly(NIPAM-TEGDMA)-40 hydrogels in 0.1N NaOH.

FIG. 3 graphically illustrates DSC thermograms of the polyNIPAM, polyNIPAM-20 and polyNIPAM-40 hydrogels.

FIG. 4 Graphically illustrates the swelling (%) of polyNIPAM, polyNIPAM-20, and polyNIPAM-40 hydrogels as a function of temperature.

FIG. 5 are photographs of polyNIPAM-40 hydrogel disks swollen in water at various temperatures.

FIG. 6 graphically illustrates the storage modulus, G′, of polyNIPAM-20 and polyNIPAM-40 hydrogels as a function of temperature at a frequency of 1 Hz and 10% shear strain.

FIGS. 7A and 7B graphically illustrate the degradation of polyNIPAM-20 and polyNIPAM-40 in 0.007N NaOH at 25° C. (7A) and 37° C. (7B).

FIG. 8 schematically depicts a representative method of forming a scaffold using sphere-templated fabrication of a fully biodegradable pNIPAM-based scaffold.

FIG. 9 is a micrograph SEM image of a pNIPAM-based scaffold with 55±5 μm pore diameter.

FIGS. 10A and 10B are light microscope images of microscope images of polyNIPAM-40-based scaffold's cross-section at 25 (A) and 37° C. (B) with pore size of 36±2 and 29±1 μm, respectively. Scale bar is 100 μm.

FIG. 11 are bar graphs illustrating cytotoxicity of a representative pNIPAM-based scaffold with 55±5 μm pore diameter and its degradation products at 5, 10, and 15 mg/mL. TCPS and latex were used as negative and positive controls, respectively. Absorbance of the samples is normalized to the TCPS.

FIGS. 12A and 12B are SEM micrographs of the scaffold surface with fibroblast cells after two days of culture (12A, scale bar from top to bottom is 100, 10, and 10 μm) and five days of culture (12B, scale bar from top to bottom is 50, 20, and 10 μm).

FIG. 13 is a composite of light microscope images of histological section of NIH3T3 cells loaded into pNIPAM-based scaffold with 55±5 μm pore diameter.

FIGS. 14A-14D are SEM micrographs of four different areas of a cell-loaded polyNIPAM-40-based scaffold cross section with a pore diameter of 55±5 μm (at 25 C).

FIG. 15 is a composite of micrographs showing SEM images of a pNIPAM-based scaffold, formed using salt leaching, with a 40±10 μm pore diameter.

FIG. 16 is a schematic diagram illustrating the capture of a cell by a scaffold that is subjected to an effective stimulus in accordance with the embodiments provided herein. The cell can optionally be released by the scaffold by applying a negative effective stimulus.

### DETAILED DESCRIPTION

The embodiments described herein include porous scaffolds formed from a stimuli-responsive polymer. The stimuli-responsive polymer of the scaffold creates a “smart” or “adaptive” scaffold that changes properties in response to an effective stimulus applied to the stimuli-responsive polymer. In a preferred embodiment, an effective stimulus applied to the scaffold initiates a phase transition event in the stimuli-responsive polymer that results in a change in the volume of the pores of the scaffold. The scaffolds can be used to capture appropriately-sized objects (e.g., cells) by using the volume-change properties of the pores. Relatedly, the scaffolds can be used as tissue-engineering scaffolds by capturing cells in the pores and introducing the cell-loaded scaffold into a cell-growth environment (e.g., in vivo).

In one aspect, a scaffold is provided. In one embodiment, the scaffold is formed from a stimuli-responsive polymer, the scaffold having a plurality of interconnected pores that each has a volume that changes in relation to a phase transition event, wherein the phase transition event is initiated by an effective stimulus to the stimuli-responsive polymer.

The scaffolds of the embodiments provided herein are porous and, thus, comprised of a plurality of interconnected pores. As used herein, the term “pores” refers to a cavity within the scaffold (e.g., a hollow space). The pores are interconnected, such that each pore is in fluid communication with at least one adjacent pore. Pores on the margins of the scaffold are particularly open to the environment surrounding the scaffold, although it will be appreciated that because the pores of the scaffold are in fluid communication with adjacent pores, liquids or gasses from outside the scaffold can penetrate to pores within the scaffold (i.e., pores not on the margins of the scaffold).

The pores of the scaffold have a shape dependant on the method by which the pores are formed. The scaffold can be formed in certain embodiments by templating the stimuli-responsive polymer onto a fused-sphere template comprising a packed plurality of spheres that are then dissolved from within the stimuli-responsive polymer to form the pores of the scaffold. In such embodiments, the pores of the scaffold are substantially spherical, although it will be appreciated that some warping and distortion of the pores may result from the dissolution of the spheres from within the stimuli-responsive polymer. Additionally, it will be appreciated that other methods used to form the scaffold may result in different shapes of the pores, and different degrees of connectivity between the pores. Other representative examples of methods for forming the scaffolds include salt leaching, salt leaching/gas foaming, phase separated material systems, electrospinning, and other templating techniques that can be used as alternatives to the sphere-templating described above.

In one embodiment, the pores have a shape selected from the group consisting of circles and ovals. Pores shaped as circles, ovals, or combinations thereof, typically result when a sphere-templating technique is used to form the scaffold.

In one embodiment, the pores have a dimension from 10 μm to 500 μm. The dimension of a pore is measured in the traditional sense (diameter) if the pore is spherical. However, if the pore is irregular in shape, the dimension of such a pore is the smallest dimension measured from diametrically opposed sides of the pore.

In certain embodiments, the pores have an irregular shape, meaning that they are not spherical, and may have variation in shape from pore to pore. Such irregular pores are typically formed in scaffolds made with a salt leaching/gas foaming process. The measurement of a pore size for such irregular pores can be measured as the mean pore size as measured across a representative population of pores.

In a particularly preferred embodiment, the pore size is from 30 μm to 50 μm. Evidence suggests that pores of about 40 μm in diameter are particularly capable of accepting and growing new tissue from cells loaded into such pores. Enhanced healing and integration of the material into a body is experienced when pore size of a scaffold is about 35-40 μm. However, it is difficult to load typical cells into 35-40 μm pores, and therefore, the volume-change capabilities of the provided scaffolds allows for 40 μm pores to be used with biocompatible scaffolds in a way such that the 40 μm pores are expanded due to an effective stimulus delivered to the scaffold that expands the pores to such a size that cells can be transported into the pores. A negative effective stimulus is then applied to the scaffold, and the pores close to about 40 μm with the cells embedded within the pores. Such a mechanism will be discussed further in more detail below.

The volume of the pores of the scaffold, and thus the scaffold itself, changes volume in relation to a phase transition event. The phase transition event can be any event that is initiated by an effective stimulus to the stimuli-responsive polymer that forms the scaffold. An effective stimulus to the stimuli-responsive polymer is one that changes the degree to which the stimuli-responsive polymer is hydrophilic. Such an effective stimulus is typically caused by the chemical makeup of the stimuli-responsive polymer, as described in more detail below. Typical stimuli include temperature, pH, and light.

In an exemplary embodiment, the stimuli-responsive polymer is a temperature-responsive polymer that becomes less hydrophilic at elevated temperatures. Accordingly, a scaffold formed from the temperature-responsive stimuli-responsive polymer will demonstrate a shrinking in pore volume as the temperature of an aqueous solution in which the scaffold is placed is raised to such a degree that the temperature of the solution becomes an effective stimulus so as to initiate a phase transition to create a less hydrophilic scaffold.

In a preferred embodiment, the stimuli-responsive polymer is a temperature-responsive polymer and the phase transition event is initiated by changing the temperature of a solution in which the scaffold is immersed, wherein raising the temperature of the solution from a first temperature that is below the phase transition temperature to a second temperature that is above the volume phase transition temperature results in a shrinking of the volume of the pores of the scaffold.

The stimuli-responsive polymer (also referred to herein as a “smart polymer”) can be any polymer having a stimuli-responsive property. The stimuli-responsive polymer can be any one of a variety of polymers that change their associative properties (e.g., change from hydrophilic to hydrophobic) in response to a stimulus. The stimuli-responsive polymer responds to changes in external stimuli such as the temperature, pH, light, photo-irradiation, exposure to an electric and/or magnetic field, ionic strength, the concentration of certain chemicals (e.g., salt or calcium concentration), and combinations thereof, by exhibiting a property change. For example, a thermally-responsive polymer is responsive to changes in temperature by exhibiting a lower critical solution temperature (LCST) in aqueous solution. The stimuli-responsive polymer can be a multi-responsive polymer, where the polymer exhibits property change in response to combined simultaneous or sequential changes in two or more external stimuli.

The stimuli-responsive polymers may be synthetic or natural polymers that exhibit reversible conformational or physio-chemical changes such as folding/unfolding transitions, reversible precipitation behavior, or other conformational changes to in response to stimuli, such as to changes in temperature, light, pH, or ions. Representative stimuli-responsive polymers include temperature-sensitive polymers (also referred to herein as “temperature-responsive polymers” or “thermally-responsive polymers”), pH-sensitive polymers (also referred to herein as “pH-responsive polymers”), and light-sensitive polymers (also referred to herein as “light-responsive polymers”).

Stimuli-responsive polymers useful in making the scaffolds described herein can be any that are sensitive to a stimulus that causes significant conformational changes in the polymer. Illustrative polymers include temperature-, pH, ion- and/or light-sensitive polymers. Hoffman, A. S., “Intelligent Polymers in Medicine and Biotechnology”, Artif. Organs. 19:458-467, 1995; Chen, G. H. and A. S. Hoffman, “A New Temperature- and Ph-Responsive Copolymer for Possible Use in Protein Conjugation”, Macromol. Chem. Phys. 196:1251-1259. 1995; Irie, M. and D. Kungwatchakun, “Photoresponsive Polymers. Mechanochemistry of Polyacrylamide Gels Having Triphenylmethane Leuco Derivatives”, Makromol. Chem., Rapid Commun. 5:829-832, 1985; and Irie, M., “Light-induced Reversible Conformational Changes of Polymers in Solution and Gel Phase”, ACS Polym. Preprints, 27(2):342-343, 1986; which are incorporated by reference herein.

Stimuli-responsive oligomers and polymers useful in the scaffolds described herein can be synthesized that range in molecular weight from about 1,000 to 30,000 Daltons. In one embodiment, these syntheses are based on the chain transfer-initiated free radical polymerization of vinyl-type monomers, as described by (1) Tanaka, T., “Gels”, Sci. Amer. 244:124-138. 1981; (2) Osada, Y. and S. B. Ross-Murphy, “Intelligent Gels”, Sci. Amer, 268:82-87, 1993; (3) Hoffman, A. S., “Intelligent Polymers in Medicine and Biotechnology”, Artif. Organs 19:458-467, 1995; also Macromol. Symp. 98:645-664, 1995; (4) Feijen, J., et al., “Thermosensitive Polymers and Hydrogels Based on N-isopropylacrylamide”, 11th European Conf. on Biomtls: 256-260, 1994; (5) Monji, N. and A. S. Hoffman, “A Novel Immunoassay System and Bioseparation Process Based on Thermal Phase Separating Polymers”, Appl. Biochem. and Biotech. 14:107-120, 1987; (6) Fujimura, M., T. Mori and T. Tosa, “Preparation and Properties of Soluble-Insoluble Immobilized Proteases”, Biotech. Bioeng. 29:747-752, 1987; (7) Nguyen, A. L. and J. H. T. Luong, “Synthesis and Applications of Water-Soluble Reactive Polymers for Purification and Immobilization of Biomolecules”, Biotech. Bioeng. 34:1186-1190, 1989; (8) Taniguchi, M., et al., “Properties of a Reversible Soluble-Insoluble Cellulase and Its Application to Repeated Hydrolysis of Crystalline Cellulose”, Biotech. Bioeng. 34:1092-1097, 1989; (9) Monji, N., et al., “Application of a Thermally-Reversible Polymer-Antibody Conjugate in a Novel Membrane-Based Immunoassay”, Biochem. and Biophys. Res. Comm. 172:652-660, 1990; (10) Monji, N. C. A. Cole, and A. S. Hoffman, “Activated, N-Substituted Acrylamide Polymers for Antibody Coupling: Application to a Novel Membrane-Based Immunoassay”, J. Biomtls. Sci. Polymer Ed. 5:407-420, 1994; (11) Chen, J. P. and A. S. Hoffman, “Polymer-Protein Conjugates: Affinity Precipitation of Human IgG by Poly(N-Isopropyl Acrylamide)-Protein A Conjugates”, Biomtls. 11:631-634, 1990; (12) Park, T. G. and A. S. Hoffman, “Synthesis and Characterization of a Soluble, Temperature-Sensitive Polymer-Conjugated Enzyme, J. Biomtls. Sci. Polymer Ed. 4:493-504, 1993; (13) Chen, G. H., and A. S. Hoffman, Preparation and Properties of Thermo-Reversible, Phase-Separating Enzyme-Oligo(NIPAAm) Conjugates”, Bioconj. Chem. 4:509-514, 1993; (14) Ding, Z. L., et al., “Synthesis and Purification of Thermally-Sensitive Oligomer-Enzyme Conjugates of Poly(NIPAAm)-Trypsin”, Bioconj. Chem. 7: 121-125, 1995; (15) Chen, G. H. and A. S. Hoffman, “A New Temperature- and pH-Responsive Copolymer for Possible Use in Protein Conjugation”, Macromol. Chem. Phys. 196:1251-1259, 1995; (16) Takei, Y. G., et al., “Temperature-responsive Bioconjugates. 1. Synthesis of Temperature-Responsive Oligomers with Reactive End Groups and their Coupling to Biomolecules”, Bioconj. Chem. 4:42-46, 1993; (17) Takei, Y. G., et al., “Temperature-responsive Bioconjugates. 2. Molecular Design for Temperature-modulated Bioseparations”, Bioconj. Chem. 4:341-346, 1993; (18) Takei, Y. G., et al., “Temperature-responsive Bioconjugates. 3. Antibody-Poly(N-isopropylacrylamide) Conjugates for Temperature-Modulated Precipitations and Affinity Bioseparations”, Bioconj. Chem. 5:577-582, 1994; (19) Matsukata, M., et al., “Temperature Modulated Solubility-Activity Alterations for Poly(N-Isopropylacrylamide)-Lipase Conjugates”, J. Biochem. 116:682-686, 1994; (20) Chilkoti, A., et al., “Site-Specific Conjugation of a Temperature-Sensitive Polymer to a Genetically-Engineered Protein”, Bioconj. Chem. 5:504-507, 1994; and (21) Stayton, P. S., et al., “Control of Protein-Ligand Recognition Using a Stimuli-Responsive Polymer”, Nature 378:472-474, 1995.

The stimuli-responsive polymers useful herein include homopolymers and copolymers having stimuli-responsive behavior. Other suitable stimuli-responsive polymers include block and graft copolymers having one or more stimuli-responsive polymer components. A suitable stimuli-responsive block copolymer may include, for example, a temperature-sensitive polymer block, or a pH-sensitive block. A suitable stimuli-responsive graft copolymer may include, for example, a pH-sensitive polymer backbone and pendant temperature-sensitive polymer components, or a temperature-sensitive polymer backbone and pendant pH-sensitive polymer components.

The stimuli-responsive polymer can include a polymer having a balance of hydrophilic and hydrophobic groups, such as polymers and copolymers of N-isopropylacrylamide. An appropriate hydrophilic/hydrophobic balance in a vinyl type polymer is achieved, for example, with a pendant hydrophobic group of about 2-6 carbons that hydrophobically bond with water, and a pendant polar group such as an amide, acid, amine, or hydroxyl group that H-bond with water. Other polar groups include sulfonate, sulfate, phosphate and ammonium ionic groups. Preferred embodiments are for 3-4 carbons (e.g., propyl, isopropyl, n-butyl, isobutyl, and t-butyl) combined with an amide group (e.g. PNIPAAm), or 2-4 carbons (e.g., ethyl, propyl, isopropyl, n-butyl, isobutyl, and t-butyl) combined with a carboxylic acid group (e.g., PPAA). There is also a family of smart A-B-A (also A-B-C) block copolymers of polyethers, such as PLURONIC polymers having compositions of PEO-PPO-PEO, or polyester-ether compositions such as PLGA-PEG-PLGA. In one embodiment, the stimuli-responsive polymer is a temperature responsive polymer, poly(N-isopropylacrylamide) (PNIPAAm).

In one embodiment, the scaffold becomes less hydrophilic after the phase transition event. PNIPAAM is an example of a stimuli-responsive polymer that becomes less hydrophilic (i.e., more hydrophobic) in response to a stimulus (e.g., temperature change relative to LCST).

In one embodiment, the stimuli-responsive polymer is a hydrogel prior to the phase transition event. Relatedly, in one embodiment, the stimuli-responsive polymer is not a hydrogel after the phase transition event.

The stimuli-responsive polymer useful in the invention can be a smart polymer having different or multiple stimuli responsivities, such as homopolymers responsive to pH or light. Block, graft, or random copolymers with dual sensitivities, such as pH and temperature, light and temperature, or pH and light, may also be used. Illustrative embodiments of the many different types of thermally-responsive polymers are polymers and copolymers of N-isopropyl acrylamide (NIPAAm). PolyNIPAAm is a thermally-responsive polymer that precipitates out of water at 32° C., which is its lower critical solution temperature (LCST), or cloud point (Heskins and Guillet, J. Macromol. Sci.-Chem. A2:1441-1455, 1968). When polyNIPAAm is copolymerized with more hydrophilic comonomers such as acrylamide, the LCST is raised. The opposite occurs when it is copolymerized with more hydrophobic comonomers, such as N-t-butyl acrylamide. Copolymers of NIPAAm with more hydrophilic monomers, such as AAm, have a higher LCST, and a broader temperature range of precipitation, while copolymers with more hydrophobic monomers, such as N-t-butyl acrylamide, have a lower LCST and usually are more likely to retain the sharp transition characteristic of PNIPAAm (Taylor and Cerankowski, J. Polymer Sci. 13:2551-2570, 1975; Priest et al., ACS Symposium Series 350:255-264, 1987; and Heskins and Guillet, J. Macromol. Sci.-Chem. A2:1441-1455, 1968, the disclosures of which are incorporated herein). Copolymers can be produced having higher or lower LCSTs and a broader temperature range of precipitation.

Synthetic pH-responsive polymers useful in making the scaffolds described herein are typically based on pH-sensitive vinyl monomers, such as acrylic acid (AAc), methacrylic acid (MAAc) and other alkyl-substituted acrylic acids such as ethylacrylic acid (EAAc), propylacrylic acid (PAAc), and butylacrylic acid (BAAc), maleic anhydride (MAnh), maleic acid (MAc), AMPS (2-acrylamido-2-methyl-1-propanesulfonic acid), N-vinyl formamide (NVA), N-vinyl acetamide (NVA) (the last two may be hydrolyzed to polyvinylamine after polymerization), aminoethyl methacrylate (AEMA), phosphoryl ethyl acrylate (PEA) or methacrylate (PEMA). pH-Responsive polymers may also be synthesized as polypeptides from amino acids (e.g., polylysine or polyglutamic acid) or derived from naturally-occurring polymers such as proteins (e.g., lysozyme, albumin, casein), or polysaccharides (e.g., alginic acid, hyaluronic acid, carrageenan, chitosan, carboxymethyl cellulose) or nucleic acids, such as DNA. pH-Responsive polymers usually contain pendant pH-sensitive groups such as —OPO(OH)₂, —COOH, or —NH₂ groups. With pH-responsive polymers, small changes in pH can stimulate phase-separation, similar to the effect of temperature on solutions of PNIPAAm (Fujimura et al. Biotech. Bioeng. 29:747-752 (1987)). By randomly copolymerizing a thermally-sensitive NIPAAm with a small amount (e.g., less than 10 mole percent) of a pH-sensitive comonomer such as AAc, a copolymer will display both temperature and pH sensitivity. Its LCST will be almost unaffected, sometimes even lowered a few degrees, at pHs where the comonomer is not ionized, but it will be dramatically raised if the pH-sensitive groups are ionized. When the pH-sensitive monomer is present in a higher content, the LCST response of the temperature-sensitive component may be “eliminated” (e.g., no phase separation seen up to and above 100° C.).

Graft and block copolymers of pH and temperature-sensitive monomers can be synthesized that retain both pH and temperature transitions independently. Chen, G. H., and A. S. Hoffman, Nature 373:49-52, 1995. For example, a block copolymer having a pH-sensitive block (polyacrylic acid) and a temperature-sensitive block (PNIPAAm) can be useful in the invention.

Light-responsive polymers usually contain chromophoric groups pendant to or along the main chain of the polymer and, when exposed to an appropriate wavelength of light, can be isomerized from the trans to the cis form, which is dipolar and more hydrophilic and can cause reversible polymer conformational changes. Other light sensitive compounds can also be converted by light stimulation from a relatively non-polar hydrophobic, non-ionized state to a hydrophilic, ionic state.

In the case of pendant light-sensitive group polymers, the light-sensitive dye, such as aromatic azo compounds or stilbene derivatives, may be conjugated to a reactive monomer (an exception is a dye such as chlorophyllin, which already has a vinyl group) and then homopolymerized or copolymerized with other conventional monomers, or copolymerized with temperature-sensitive or pH-sensitive monomers using the chain transfer polymerization as described above. The light sensitive group may also be conjugated to one end of a different (e.g., temperature) responsive polymer. A number of protocols for such dye-conjugated monomer syntheses are known.

Although both pendant and main chain light sensitive polymers may be synthesized and are useful for the methods and applications described herein, the preferred light-sensitive polymers and copolymers thereof are typically synthesized from vinyl monomers that contain light-sensitive pendant groups. Copolymers of these types of monomers are prepared with “normal” water-soluble comonomers such as acrylamide, and also with temperature- or pH-sensitive comonomers such as NIPAAm or AAc.

Light-sensitive compounds may be dye molecules that isomerize or become ionized when they absorb certain wavelengths of light, converting them from hydrophobic to hydrophilic conformations, or they may be other dye molecules that give off heat when they absorb certain wavelengths of light. In the former case, the isomerization alone can cause chain expansion or collapse, while in the latter case the polymer will precipitate only if it is also temperature-sensitive.

Light-responsive polymers usually contain chromophoric groups pendant to the main chain of the polymer. Typical chromophoric groups that have been used are the aromatic diazo dyes (Ciardelli, Biopolymers 23:1423-1437, 1984; Kungwatchakun and Irie, Makromol. Chem., Rapid Commun. 9:243-246, 1988; Lohmann and Petrak, CRC Crit. Rev. Therap. Drug Carrier Systems 5:263, 1989; Mamada et al., Macromolecules 23:1517, 1990, each of which is incorporated herein by reference). When this type of dye is exposed to 350-410 nm UV light, the trans form of the aromatic diazo dye, which is more hydrophobic, is isomerized to the cis form, which is dipolar and more hydrophilic, and this can cause polymer conformational changes, causing a turbid polymer solution to clear, depending on the degree of dye-conjugation to the backbone and the water solubility of the main unit of the backbone. Exposure to about 750 nm visible light will reverse the phenomenon. Such light-sensitive dyes may also be incorporated along the main chain of the backbone, such that the conformational changes due to light-induced isomerization of the dye will cause polymer chain conformational changes. Conversion of the pendant dye to a hydrophilic or hydrophobic state can also cause individual chains to expand or contract their conformations. When the polymer main chain contains light sensitive groups (e.g., azo benzene dye) the light-stimulated state may actually contract and become more hydrophilic upon light-induced isomerization. The light-sensitive polymers can include polymers having pendant or backbone azobenzene groups.

Polysaccharides, such as carrageenan, that change their conformation, for example, from a random to an ordered conformation, as a function of exposure to specific ions, such as potassium or calcium, can also be used as the stimulus-responsive polymers. In another example, a solution of sodium alginate may be gelled by exposure to calcium. Other specific ion-sensitive polymers include polymers with pendant ion chelating groups, such as histidine or EDTA.

In preferred embodiments, the stimuli-responsive polymer comprises polymers selected from the group consisting of poly (N-isopropyl acrylamide) (variously abbreviated herein as PNIPA, PNIPAAm, PNIPAA, PNIPAm or PNIPAM), poly methacrylic acid (PMAA), poly(ethylene oxide)-poly(propylene oxide)-poly(ethylene oxide) (PEO-PPO-PEO) (also known as Poloxamers or Pluronics), poly(ethylene glycol)/polyester copolymers, poly(vinylpyrrolidone)-based polymers, elastin peptides, and natural modified polymers, such as methylcellulose, chitosan, and xyloglucan, and combinations thereof.

Biodegradable Scaffolds

In certain embodiments, the scaffold is a biodegradable scaffold. As used herein, the term “biodegradable” refers to materials that will biodegrade in vivo to yield an oligomeric compound that can be excreted or otherwise biologically cleared from the body.

Because the scaffold may comprise several polymers, or may comprise crosslinked polymers and copolymers, several routes to a biodegradable scaffold are possible. Of particular interest are biodegradable scaffolds wherein the stimuli-responsive polymer comprises a polymer backbone that is a copolymer that includes both a stimuli-responsive polymer portion and a biodegradable polymer portion. Such an embodiment provides a biodegradable polymer backbone that will biodegrade at the biodegradable polymer portions to yield oligomeric stimuli-responsive polymer portions.

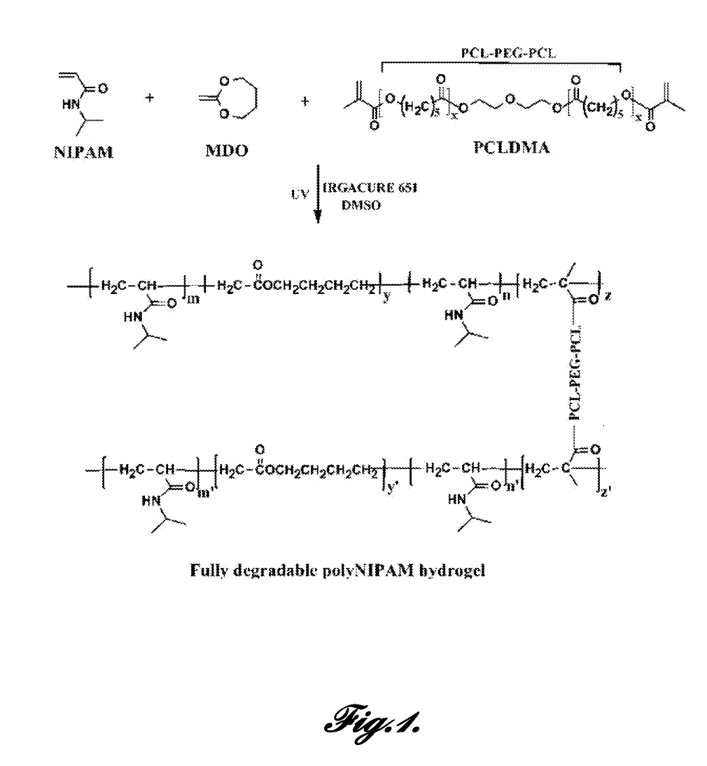

As illustrated below in Example 1 (see, e.g., FIG. 1), a polyNIPAM stimuli-responsive polymer is used to form a biodegradable scaffold in accordance with the embodiments provided herein. The polyNIPAM is the primary polymer used in the scaffold, but the polymer backbone is synthesized as a copolymer with 2-methylene-1,3-dioxepane (MDO), which yields an ester linkage in the polymer backbone that is biodegradable.

In one embodiment, the biodegradable portion comprises a moiety selected from the group consisting of an ester, an amide, a phosphazine, an anhydride, an orthoester, and a disulfide.

In one embodiment, the biodegradable portion is selected from the group consisting of oligo or polycaprolactone (PCL)-based segments, PCL-PEG-PCL, PLA-PEG-PLA, PLGA-PEG-PLGA, PCL-PEG-PPG-PCL, and combinations thereof.

In certain embodiments, the scaffold is biodegradable because the crosslinkers that crosslink the stimuli-responsive polymer backbones are biodegradable. Biodegrading such a polymer yields degraded (e.g., broken) crosslinkers and intact polymer backbones. In additional embodiments, both biodegradable crosslinkers and biodegradable copolymer units in the polymer backbone are incorporated into the same stimuli-responsive polymer scaffold. Such a combination yields scaffolds that are biodegradable at two sites, the crosslinker and the polymer backbone, to yield superior biodegradability. For example, as set forth in the EXAMPLES below, a fully biodegradable polyNIPAAm-based scaffold can be formed using NIPAAm copolymerized with MDO that contributes degradable PCL portions to polyNIPAAm backbone, and crosslinked with biodegradable diacrylated PCL-PEG-PCL.

With regard to the biodegradable crosslinkers, in one embodiment, the stimuli-responsive polymer comprises a plurality of biodegradable crosslinking moieties that crosslink stimuli-responsive polymer.

In one embodiment, the biodegradable crosslinking moieties are selected from the group consisting of derivatives of poly(ε-caprolactone) (PCL), polylactic acid, dextran, chitosan, disulfide, amino-acid, polyaspartic acid, and combinations thereof.

In one embodiment, the biodegradable crosslinking moieties comprise a moiety selected from the group consisting of an ester, an amide, a phosphazine, an anhydride, an orthoester, a polyhydroxyalkanoate, and a disulfide.

In one embodiment, the biodegradable crosslinking moieties will biodegrade in vivo to yield oligomeric units that can be excreted. In one embodiment, the oligomeric units comprise the stimuli-responsive polymer.

Angiogenic Scaffolds

In certain embodiments, the scaffold is used for tissue engineering. Accordingly, in one embodiment, the scaffold is angiogenic. As used herein, the term “angiogenic” means a scaffold that encourages vascularized integration of the material into a hosting tissue through increased re-growth and development of blood vessels and reduced fibrosis. As will be described further below, the scaffolds can be used to capture cells and facilitate tissue engineering. It will be appreciated that the properties of the scaffold can be tailored to provide an appropriate environment to encourage cell growth. Because different types of cells require different preferred environments in which to grow, appropriate modification of the scaffolds to accommodate any such cells is contemplated by the embodiments provided herein.

Methods for Forming the Scaffold

In another aspect, a method is provided for forming a scaffold as provided herein. In one embodiment, the method comprises the steps of:

(a) providing a template comprising a plurality of packed particles in a vessel, said packed particles being packed in an arrangement such that voids in between the particles can be infiltrated by a liquid poured into the vessel;

(b) filling the voids in between the particles with a solution comprising a monomer for the stimuli-responsive polymer and a polymerization initiator;

(c) polymerizing the monomer for the stimuli-responsive polymer using the polymerization initiator to provide the stimuli-responsive polymer as a matrix surrounding the packed particle template, thereby providing a particle-scaffold composite; and

(d) dissolving the packed particles from the particle-scaffold composite to provide the scaffold.

The method provides a fabrication route for the stimuli-responsive scaffolds disclosed herein. In the first step of the method, a template is provided that comprises a plurality of packed particles in a vessel. The vessel can be any container suitable to hold the packed particles so as to maintain the packing of the particles and to contain any liquids that are added as required by the method (e.g., polymer precursor solutions). The packed particles can be any particles known to those of skill in the art, such as spherical (e.g., polymer spheres as disclosed in Example 1) or other geometrically shaped particles, or salt-based particles such as the sodium chloride particles used in the salt-leaching technique described herein in Example 2.

The packed particles are packed such that voids in between the particles can be infiltrated by a liquid poured into the vessel. Particularly, polymer precursor liquids (e.g., monomers and initiators) are poured into the vessel, and the liquids infiltrate the voids between the packed particles to provide a network that eventually become the stimuli-responsive polymer scaffold at the conclusion of the method. The shape of the voids essentially becomes the shape of the scaffold around the pores in the finished stimuli-responsive scaffold.

In the next step of the method, the voids in between the particles are filled with a solution that includes a monomer for the stimuli-responsive polymer and a polymerization initiator. The stimuli-responsive polymer is useful in the embodiments described herein have been described above, and those of skill in the art will appreciate the synthesis of such polymers, particularly through the use of a polymerization initiator.

After the voids in between the packed particles have been filled with the monomer and initiator, the two components are polymerized within the voids to provide a stimuli-responsive polymer as a matrix surrounding the packed particle template (referred to herein as a “particle-scaffold composite”).

The method concludes with a step of dissolving the packed particles from within the particle-scaffold composite to provide the scaffold. An appropriate solvent will dissolve, or otherwise solvate, the plurality of packed particles so as to allow the solvated particles to exit the scaffold. The solvated particle material exits the scaffold and leaves only the stimuli-responsive polymer scaffold as the remaining element of the particle-scaffold composite. Upon removal from the dissolving solution, the scaffold of the invention is provided.

It will be appreciated that the polymer may be a traditional single-chain polymer (e.g., comprising stimuli-responsive polymer portions, and optionally, biodegradable portions), although in certain embodiments, a crosslinking moiety is added so as to form a crosslinked stimuli-responsive polymer material (e.g., a hydrogel). If a crosslinking element of the polymer of the scaffold is desired, the method further includes the introduction of a monomer with a crosslinking moiety to the solution comprising the stimuli-responsive polymer monomer and the polymerization initiator. Furthermore, the step of polymerizing the monomer for the stimuli-responsive polymer using the polymerization initiator further comprises polymerizing a monomer with the crosslinking moiety to provide a crosslinked stimuli-responsive polymer as a matrix surrounding the packed particle template.

The particles may be packed in any number of configurations, said configurations depending largely on the shape of the particles and the packing method.

One preferred approach to sphere-templating was developed by Ratner et al. and is disclosed in U.S. Patent Application Publication No. 2008/0075752, the disclosure of which is incorporated herein by reference in its entirety.

In one embodiment, the packed particles are packed in a configuration selected from the group consisting of simple cubic, body-centered, face-centered cubic, irregular, and hexagonal close-packed.

In one embodiment, dissolving the packed particles comprises submerging the particle-scaffold composite in a solvent for the particles.

In one embodiment, the method further comprises a step of heating the template to a temperature sufficient to sinter the particles together prior to filling the voids in between the particles.

In one embodiment, the particles are a material selected from the group consisting of poly(methyl methacrylate), polystyrene, sucrose, sodium chloride, and poly(ethyl methacrylate).

In one embodiment, the particles are monodisperse spheres having a size of from 10 micrometers to 500 micrometers.

While in one preferred embodiment the packed particles are packed spheres that are sintered together and then dissolved with an appropriate solvent after scaffold formation, in other embodiments alternative techniques such as salt leaching/gas foaming and phase segregation (copolymer phase segregation or immiscible material phase segregation) can be used.

In one embodiment, salt leaching or salt leaching/gas foaming is used to provide the scaffold. Such an embodiment is described further below in Example 2. Generally, salt leaching comprises a method where a salt and/or a gas-foaming agent are provided in tandem such that upon the application of appropriate solvents, the salt solvates and the gas-foaming agent provides a foaming action. In general, salt crystals, for example, ammonium carbonate, are mixed with organic monomer solution prior to polymerization. After a gel formation the material is exposed to water and heat to both solubilize and induce decomposition of the foaming agent that results in a release of gases, such as carbon dioxide, and ammonia and also to dissolve salt crystals. In general, by defining a size of the salt crystals one can control pore size of a scaffold and by using gas foaming interconnectivity is achieved. Weight percent of a salt as well as of a foaming agent affects scaffold's porosity. In a preferred embodiment, the salt-leaching agent is sodium chloride and the gas-foaming agent is ammonium carbonate. In such a combination, an aqueous acid and then water are used as a solvent system to clear the salt-leaching and gas-foaming agents from the formed scaffold.

In another embodiment, sodium bicarbonate salt is used and extract from the scaffold with a mildly acidic solution to generate foaming.

In one embodiment, the particles are a salt-leaching/gas foaming mixture comprising sodium chloride and ammonium carbonate and wherein dissolving the particles comprises submerging in aqueous acid and then water.

The polymerization initiator can be any initiator known to those of skill in the art. The initiator will be determined based on the nature of the polymerization required for the monomer(s) and any optional crosslinking moieties utilized in forming the scaffold.

In one embodiment, the polymerization initiator is a photoinitiator and wherein the polymerizing step of the method comprises exposing the solution to electromagnetic radiation having a wavelength and intensity sufficient to activate the photoinitiator.

Cell Growth Using the Scaffolds

In another aspect, a method is provided for growing cells using a scaffold as provided herein. In one embodiment, the scaffold has a large pore state and a small pore state depending on whether a volume phase transition event has occurred, the method comprising the steps of:

(a) contacting a suspension of cells with the scaffold in the large pore state such that the cells infiltrate the large pores of the scaffold;

(b) applying an effective stimulus to the scaffold so as to transition the scaffold to the small pore state, thereby trapping the cells in the pores;

(c) placing the scaffold containing trapped cells in a location where cell growth is desired;

(d) culturing the cells within the scaffold, which provides mechanical and biochemical support for the cells; and

(e) biodegrading the scaffold to provide space for aggregated cells or newly formed tissue.

In the method, the scaffold is a stimuli-responsive scaffold as provided herein that is formed with a stimuli-responsive polymer. In the method, the scaffold has a large pore state and a small pore state. The size of the pore (e.g., the pore state) is based on whether a volume phase transition event has occurred. For example, if a stimuli-responsive scaffold is a temperature-responsive scaffold, the scaffold may have a large pore state at room temperature and a small pore state at an elevated temperature that is above a volume phase transition temperature (VPTT). It will be appreciated that any effective stimulus is useful for transitioning between the large pore state and small pore state, and therefore, the appropriate stimulus is based on the composition of the scaffold and particularly the stimuli-responsive polymer. As is typical with stimuli-responsive polymers, the opposite of the effective stimulus (e.g., reducing the temperature below the VPTT) will provide the opposite effect (e.g., transitioning from the small pore state to the large pore state).

In the first step of the method, the scaffold is in the large pore state and is contacted with a suspension of cells such that the cells infiltrate the large pores of the scaffold.

The method continues with a step of applying an effective stimulus to the scaffold so as to transition the scaffold to the small pore state. In the small pore state, the cells are preferably trapped within the pores.

The method continues with a step of placing the scaffold containing the trapped cells in a location where cell growth is desired. Representative locations include in vitro and in vivo.

In one embodiment, the cells are selected from the group consisting of endothelial cells, fibroblasts, macrophages, smooth muscle cells, mesenchymal stem cells, hematopoetic stem cells, embryonic stem cells, hepatocytes, cardiomyocytes, neurons, keratinocytes, osteoblasts, chondrocytes, and combinations thereof.

The method continues with the step of culturing the cells within the scaffold. The scaffold provides mechanical and biochemical support for the cells, as is necessary for the cells to grow and replicate. In one embodiment, culturing the cells within the scaffold comprises enhanced angiogenesis and decreased fibrosis. In a further embodiment, culturing the cells provides vascularized tissue at the location of the scaffold.

The method concludes with a step of biodegrading the scaffold to provide space for aggregated cells or newly formed tissue. In this step, the scaffold biodegrades, and the cells aggregate and form new tissue. The scaffold biodegrades through the degradation of biodegradable moieties in the polymer backbone, crosslinkers, or both, as described above. Biodegrading the scaffold makes way for newly formed tissue, and the tissue grows in the location in which the scaffold was replaced. Eventually, no remaining aspects of the scaffold remain in the location, and the newly formed tissue takes its place.

The use of the scaffolds for capturing cells (or other particles) may be better understood with reference to FIG. 16. In FIG. 16, a stimuli-responsive scaffold in a large pore state 100 is provided. The scaffold 100 includes a main pore 102 into which a cell 120 will be loaded in this exemplary embodiment. Also illustrated in the scaffold 100 are adjacent pores 104 that are fluidly connected with main pore 102 through channels 106. Finally, the scaffold 100 includes exterior pore walls 108 are separated by a distance d₁ of sufficient length to allow a cell 120 to infiltrate the pore 102.

The scaffold 100 is loaded with a cell suspension and a cell 120 infiltrates the pore 102.

In order to trap the cell 120 in the scaffold 100, a phase change event (Δ) is initiated by providing an effective stimulus to the scaffold (e.g., temperature is raised above a VPTT). After the phase change event, the large pore state scaffold 100 becomes a small pore state scaffold 200 that includes pore walls 208 separated by an opening having a length d₂ that is smaller than the diameter of the cell 120. Accordingly, the cell 120 is trapped in the scaffold 200.

When a cell 120 is trapped in the scaffold 200, the scaffold 200 may then be placed into an appropriate location where cell growth is desired. Therefore, this exemplary method may end with the cell 120 being trapped in the small pore state scaffold 200.

Optionally, if release of the cell 120 at a particular location is desired, a negative phase change (−Δ) event (e.g., lower the temperature below the VPTT) can be applied to the small pore state scaffold 200 to provide a large pore state scaffold 100 again such that the cell 120 can then be released. Such a method step may be useful to release trapped cells from within the scaffold at the specific location where the scaffold was implanted (e.g., for delivery of cells to a specific site in vivo).

In another aspect, a method for cell growth is provided. The method of this aspect is similar to that described above, but only the steps of contacting a suspension of cells, applying an effective stimulus, and placing the scaffold containing trapped cells in the location, are encompassed by the method. Accordingly, in this aspect, culturing the cells and biodegrading the scaffold are not encompassed.

In one embodiment, the scaffold has a large pore state and a small pore state depending on whether a volume phase transition event has occurred, the method comprising the steps of:

(a) contacting a suspension of cells with the scaffold in the large pore state such that the cells infiltrate the large pores of the scaffold;

(b) applying an effective stimulus to the scaffold so as to transition the scaffold to the small pore state, thereby trapping the cells in the pores; and

(c) placing the scaffold containing trapped cells in a location where cell growth is desired.

The following examples illustrate representative embodiments now contemplated for practicing the invention, but should not be construed to limit the invention.

### EXAMPLES

### Example 1

### Preparation of a Temperature-Sensitive Hydrogel Scaffold Using Microsphere Templating

We have developed a thermoresponsive poly(N-isopropyl acrylamide)-based scaffold with degradability and controlled porosity. Biodegradable poly(N-isopropyl acrylamide) hydrogels were synthesized by photocopolymerization of N-isopropylacrylamide with 2-methylene-1,3-dioxepane and polycaprolactone dimethacrylate. The hydrogels' phase transition temperature, swelling, and viscoelastic properties, as well as hydrolytic degradability at 25 and 37° C., were explored. A sphere-templating technique was applied to fabricate hydrogel scaffolds with controllable pore size and a highly interconnected porous structure. The scaffold pore diameter change as a function of temperature was evaluated and, as expected, pores decreased in diameter when the temperature was raised to 37° C. 3-(4,5-Dimethylthiazol-2-yl)-2,5-diphenyltetrazolium bromide (MTT) test results suggested neither the scaffolds nor their degradation products were cytotoxic to NIH3T3 cells. Scaffolds with 55±5 μm pore diameter were loaded with NIH3T3 cells and then were warmed to 37° C. entrapping cells in pores approximately 39 μM in diameter, a size range we have found to be optimal for angiogenesis and biointegration. Cells showed uniform infiltration and an elongated morphology after 7 days of culture. Due to the controlled monodisperse pore diameter, highly interconnected architecture, fully degradable chemistry and thermoresponsive properties, the polyNIPAM-based scaffolds developed here are attractive for applications in tissue engineering.

One purpose of this example is to demonstrate the preparation of fully biodegradable, thermo-swellable scaffolds based on polyNIPAM hydrogels with controlled pore size and a highly interconnected porous structure that have potential for tissue engineering applications.

We report a method for preparation of thermoresponsive scaffolds based on a fully degradable polyNIPAM hydrogel with a controllable, monodisperse, highly interconnected porous structure for potential applications in tissue engineering. First, polyNIPAM hydrogels with degradable units within the polymer backbone and at the cross-linking sites were synthesized by photopolymerization of NIPAM with MDO and polycaprolactone dimethacrylate (PCLDMA). The VPTT of the hydrogels prepared at different [NIPAM]/[MDO] molar ratios were measured and their swelling and viscoelastic properties as well as hydrolytic degradability at 25 and 37° C. were explored. Second, a sphere-templating technique was applied to fabricate fully degradable scaffolds, with monodisperse controlled pore size and highly interconnected architecture, based on the aforementioned polyNIPAM hydrogels. The morphology of the scaffolds with different pore sizes was characterized and change in the pore size as a function of temperature was examined. The cytotoxicity of the scaffolds and their degradation products were analyzed using the MTT assay and the adhesion of the fibroblast cells to the material was explored. To demonstrate the potential of the developed material for tissue engineering, the scaffolds were loaded with NIH3T3 cells using the thermoresponsive properties of the material and cell distribution, infiltration and morphology were investigated.

Materials. NIPAM (Aldrich, 97%), MDO (Polysciences Inc.), tetraethylene glycol dimethacrylate (TEGDMA) (Polyscience Inc.), polycaprolactone diol (Mn ˜530; Aldrich), methacryloyl chloride (Fluka, 97%), triethylamine (TEA) (Sigma, 99.5%), un-cross-linked poly(methyl methacrylate) (PMMA) microspheres (Microbeads (sphere diameter ˜38 μm); Kupa Inc. (sphere diameter ≦100 μm); Polyscience Inc. (sphere diameter ˜200 μm, M_{w}) 75 000)), DMSO anhydrous (EG) (Sigma, 99.8%), ethylene glycol anhydrous (EG; Sigma, 99.8%), 2,2-dimethoxy-2-phenylacetophenone (IRGACURE 651, Ciba-Geigy), dichloromethane (EMD Chemicals Inc., HPLC grade), acetone (EMD Chemicals Inc., HPLC grade), Vybrant MTT cell proliferation assay kit (Invitrogen), and Dulbecco's modified Eagle medium (DMEM; Invitrogen) were used.

Analytical Methods. ¹H NMR spectra were obtained with a Bruker AV-300 spectrometer. For chloroform-d, chemical shifts are expressed in ppm downfield from tetramethylsilane used as an internal standard.

Photopolymerization was accomplished with a UV lamp (PC451050, Hanovia 450-W Hg lamp, HANOVIA Specialty Lighting LLC, NJ). Distance of the lamp from polymerization mixture is 30 cm.

The scaffold and cell morphology were determined by scanning electron microscopy (SEM) (FEI SEM XL Siron, Hillsboro, Oreg.). Dry samples were Au/Pd sputter coated for 60 s (SPI Supplies, West Chester, Pa.).

Details regarding additional equipment items that were used during this research are described later in pertinent paragraphs.

Synthesis of PCLDMA. Polycaprolactone diol (Mn 530, 25 g, 0.047 mol) and TEA (19.7 mL, 0.14 mol) were dissolved in anhydrous dichloromethane (150 mL), and the solution was cooled to 0° C. Methacryloyl chloride (13.8 mL, 0.14 mol) was added dropwise, and the reaction mixture was stirred at room temperature (RT) for 18 h. The formed solid was filtered off, and the dichloromethane solution was washed with 0.1N HCl, saturated NaHCO₃ and H₂O. The organic phase was dried over MgSO₄, filtered and evaporated to produce an orange oil. Purified PCLDMA was obtained by precipitation into cold hexanes as a slightly yellow wax (yield 82%). The degree of methacrylation was determined with ¹H NMR spectroscopy by ratio between the integral area under the peak at δ 6.12 (s, 2H, olefinic, cis) and 3.70 (m, 4H, —OCH₂CH₂OCH₂CH2O—).

Synthesis of PolyNIPAM-20 and PolyNIPAM-40 Hydrogels. Fully degradable polyNIPAM-20 and polyNIPAM-40 hydrogels were prepared by photocopolymerization of NIPAM with a 20 and 40 molar percent of MDO monomer, respectively, as shown in FIG. 1.

In a typical experiment, NIPAM (0.9 g, 0.008 mol), PCLDMA (0.158 g, 2.4×10⁻⁴ mol, 3% mol/mol), MDO (0.181 mL, 1.6×10⁻³ mol (20% mol/mol) or 0.362 mL, 3.2×10⁻³ mol (40% mol/mol)), and photoinitiator 2,2-dimethoxy-2-phenylacetophenone (25 mg, 8×10⁻⁵ mol (1% mol/mol)) were dissolved in anhydrous DMSO/EG solution (1.5 mL, 1:1 u/u). The reaction mixture was then cast into a mold (consisting of two microscope slides separated by 1 mm thick Teflon spacer) and photopolymerized for 3 min. The polymer sheet was then removed from the mold and washed with acetone and water to extract residues of unreacted reagents.

For NMR, linear polyNIPAM-40 was synthesized according the procedure described above but in the absence of PCLMDA cross-linker. After polymerization the crude linear material was dissolved in chloroform, precipitated from cold ether and dried under vacuum to obtain purified linear polyNIPAM-40 as a white powder.

Synthesis of Poly(NIPAM-TEGDMA)-20, Poly(NIPAM-TEGDMA)-40, and Poly(NIPAM-TEGDMA) Hydrogels. Partially degradable poly(NIPAM-TEGDMA)-20 and poly(NIPAM-TEGDMA)-40 hydrogels (with a degradable backbone but a nondegradable crosslinking sites) were prepared according the procedure described in the paragraph above but in the presence of the nondegradable cross-linker TEGDMA (79 μL, 2.4×10⁻⁴ mol, 3% mol/mol) instead of PCLDMA. Nondegradable poly(NIPAM-TEGDMA) hydrogel (with nondegradable both backbone and cross-linking site) was prepared as described herein but in the absence of MDO monomer.

Synthesis of PolyNIPAM Hydrogel. Partially degradable polyNIPAM hydrogel with a nondegradable backbone and degradable cross-linking sites was prepared according the procedure for synthesis of fully degradable hydrogels but in absence of MDO monomer.

Hydrogel Compositions Synthesized in this Work. Table 1 summarizes compositions and anticipated degradability of the hydrogels prepared in the present work.

Degradation Studies. 1. Impact of NIPAM Copolymerization with MDO on the Degradation of the PolyNIPAM Backbone. Nondegradable poly(NIPAM-TEGDMA) and partially degradable poly(NIPAMTEGDMA)-20 and poly(NIPAM-TEGDMA)-40 hydrogel sheets were punched into 8 mm disks. The disks were lyophilized to determine the original dry weight (W₁). The dry disks were then placed in 0.1 N NaOH solution, and the samples were shaken at RT. Poly(NIPAM-TEGDMA), poly(NIPAM/TEGDMA)-20 and poly(NIPAM/TEGDMA)-40 samples (triplicates) were taken at different points in time and washed with H₂O to remove residual NaOH. The samples were then lyophilized to determine dry weight after degradation (W₂). The degradation was evaluated by calculating the percent of weight loss using Equation 1:

weight loss(%))[(W₁−W₂)/W₁]100 (1)

2. Hydrolytic Degradation of Fully Degradable PolyNIPAMBased Hydrogels. Fully degradable polyNIPAM-20 and polyNIPAM-40 hydrogel sheets were punched into 8 mm disks. The disks were lyophilized to determine the original dry weight (W₁). Then, the dry disks were placed in 0.007 N NaOH solution and the samples were shaken at 25 and 37° C. For both temperatures, polyNIPAM-20 and polyNIPAM-40 samples (triplicates) were taken at different points in time, washed with H2O and lyophilized to determinate dry weight after degradation (W₂). The degradation was evaluated by calculating the percent of weight loss using Equation 1.

TABLE 1
Summary of the Hydrogels Compositions
Synthesized in this Example

| | MDO %, | degradable | |
|---|---|---|---|
| hydrogel name | mol/mol | backbone | cross-linking site |
| polyNIPAM-20 | 20 | ✓ | ✓ |
| polyNIPAM-40 | 40 | ✓ | ✓ |
| polyNIPAM | — | — | ✓ |
| poly(NIPAM-TEGDMA)-20 | 20 | ✓ | — |
| poly(NIPAM-TEGDMA)-40 | 40 | ✓ | — |
| poly(NIPAM-TEGDMA) | — | — | — |

VPTT Determination. VPTT of the polyNIPAM-20, polyNIPAM-40 and polyNIPAM hydrogels was determined by differential scanning calorimetery (DSC) (Netzsch DSC 200). The hydrogel sheets, swollen to equilibrium, were punched into 3 mm diameter disk and the excess water on the disk surface was removed with wet filter paper. Then, the samples were placed in an aluminum pan and sealed with an aluminum lid. The samples were scanned from 25 to 50° C. at a heating rate of 3° C./min under dry nitrogen. The VPTT was defined as the onset temperature of the endotherm.

Swelling Study. PolyNIPAM-20, polyNIPAM-40, and polyNIPAM hydrogel sheets were punched into 8 mm disks. The disks were lyophilized to determinate the dry weight (W_{dry}). The samples were then swollen in distilled water at 4, 25, and 37° C. for 24 h to reach the equilibrium state. For each temperature, the excess water on the swollen hydrogel surface was removed with wet filter paper and the weight of the swollen sample (W_{swollen}) was determined (in triplicate). The swelling percent was calculated using Equation 2:

swelling(%)=[(W_{swollen}−W_{dry})/W_{dry}]100 (2)

Rheology. Viscoelastic properties of polyNIPAM-20 and polyNIPAM-40 were characterized by dynamic shear oscillation measurements. The rheology study was performed on swollen hydrogel disks (25 mm diameter, 0.5 cm thickness) using a rheometer (AR-G2, TA Instruments, New Castle, Del.) with a 20 mm diameter parallel plate. The effect of the temperature on the storage modulus (G′) was determined from 25 to 45° C. with a heating rate of 1° C./min, at constant frequency and shear strain of 1 Hz and 10%, respectively. The temperature of the plate was controlled by connection to a recirculating water bath.

Sphere-Templated Scaffold Fabrication. Step 1. Template Preparation. PMMA microspheres were fractionated to the following size cuts: 35±1, 49±8, 81±8, and 188±26 μm, with an ATM model L3P Sonic Sifter. The beads were transferred to a mold (composed of two microscope slides separated by a 1 mm thick Teflon spacer) and sonicated for 10 min for optimal packing. The beads were then sintered for 20 h at 140° C. to obtain PMMA templates with neck sizes (interconnects between the beads) of 30% of the bead diameter.

Step 2. Scaffold Fabrication. PMMA templates were infiltrated with the reaction mixture composed of NIPAM (0.9 g, 0.008 mol), PCLDMA (0.158 g, 2.4×10⁻⁴ mol, 3% mol/mol), MDO (0.181 mL, 1.6×10⁻³ mol (20% mol/mol) or 0.362 mL, 3.2×10⁻³ mol (40% mol/mol)), and photoiniator 2,2-dimethoxy-2-phenylacetophenone (25 mg, 8×10⁻⁵ mol (1% mol/mol)) dissolved in anhydrous DMSO/EG solution (1.5 mL, 1:1 v/v). The mixture was photopolymerized for 5 min and the PMMA template infused with polymerized hydrogel was removed from the mold and placed in dichloromethane to dissolve the PMMA beads. The scaffold obtained, composed of a fully degradable polyNIPAM gel, was washed in acetone and then hydrated in distilled water.

Shrinkage. Swollen polyNIPAM-40-based scaffolds disks (10 mm diameter with different pore diameters) were embedded in a gel (Tissue Tek OCT Compound), frozen in liquid nitrogen and then cut to 25 μm thick sections with a Leica ultramicrotome CM1850. The sections were immersed in water and observed under an inverted optical microscope equipped with an incubation warmer (Nikon eclipse TE200). Pore diameter was measured with MetaMorph software (version 6.0, Molecular Devises, Pa.) at 25° C. (D₂₅) and 37° C. (D₃₇) and shrinkage percent was calculated by using Equation 3:

linear shrinkage(%))[(D₂₅−D₃₇)D_{25]100} (3)

volumetric shrinkage (%))[(V₂₅−V₃₇)V_{25]100}, where V=4/3π(D/2)³

Cytotoxicity. Cytotoxicity of the scaffolds and their degradation products were evaluated by using an MTT assay.

1. Scaffold Cytotoxicity. Scaffolds were sterilized with 70% ethanol, washed with sterile PBS, and placed in DMEM for 24 h. At the same time, 0.5 mL of 3T3 mouse fibroblasts (1×10⁴ cells/well) were seeded in 24-well culture plates for 24 h. The media then was removed from the wells and replaced with 0.5 mL of scaffold eluent. The cells were incubated with media eluted from the scaffold for an additional 24 h, then washed with phenol red-free DMEM media, and afterward exposed for 4 h to 0.5 mL of phenol red-free DMEM media containing 10% of MTT solution (5 mg/mL MTT reagent in PBS). A total of 1 mL of DMSO was then added to extract a blue product by vigorous pipetting. Tissue culture polystyrene (TCPS) and latex were used as the negative and positive controls, respectively, and were treated similarly as above. A 200 μL aliquot from each well was transferred into a 96-well plate and absorbance was measured at 550 nm with a microplate reader (tunable VERSAmax microplate reader, Molecular Devices, CA).

2. Degradation Products Cytotoxicity. Scaffolds were hydrolyzed in 0.1 N NaOH. The solution was neutralized and degradation products were extracted with chloroform. The organic phase was dried over MgSO₄, filtered, and chloroform was evaporated to obtain a white powder. The degradation products were dissolved into media at concentrations of 5, 10, and 15 mg/mL and cytotoxicity at different concentrations was evaluated according the procedure for the scaffold eluent, described in the paragraph above.

Cell Adhesion and Morphology. PolyNIPAM-40-based scaffold was punched into 8 mm disks, sterilized with 70% ethanol, washed with sterile PBS, and soaked in culture media at 4° C. overnight prior to seeding. Afterward, disks immersed in DMEM media were placed at 37° C. for 24 h to allow shrinkage of the material. The shrunken disks then were removed from the media and placed into a 96-well plate to fit exactly the diameter of the well and to serve as a surface for cell culture. 180 μL of cell suspension (1×10⁴ cells/well) was added and cells were cultured on the surface of the scaffold for 2 and 5 days. At each point in time the media was removed, the scaffold's surface was washed with warm sterile PBS and then fixed with 2% glutaraldehyde solution at 37° C. At the end of fixation the sample was immediately frozen in liquid nitrogen and lyophilized. The samples were observed under SEM to explore cell adhesion and morphology.

Cell Loading Study. The polyNIPAM-40-based scaffold with 55±5 μm pore diameter was punched into 10 mm disks, sterilized with 70% ethanol, washed with sterile PBS, and soaked in culture media at 4° C. overnight. Prior to loading with cells, the excess of media was removed from the scaffold's surface with wet filter paper. Afterward, the disks were placed on dry filter paper and 50 μL of the 3T3 cell suspension (1×10⁷ cells/mL) was added 4 times to the top of each disk (triplicates). The scaffolds were then placed into a 24-well plate and 2 mL of media was carefully added. The cells were cultured for 7 days and the media was changed every other day. The scaffolds were then fixed with a methanol-glacial acetic acid solution (9:10r) for 30 min at 37° C. For histological analysis, samples were dehydrated through graded ethanol/xylene at 37° C. and then embedded in paraffin. Paraffin blocks were sectioned (5 μm thick) onto charged Superfrost plus slides (VWR International, West Chester, Pa.), deparaffinized, rehydrated and stained with hematoxylin and eosin. Images were obtained with a Nikon E800 upright microscope equipped with MetaMorph software (version 6.0, Molecular Devises, Pa.).

For observation under SEM the samples' cross sections of approximately 1 mm thick were deparaffinized, rehydrated, frozen in liquid nitrogen and lyophilized.

Fully Degradable PolyNIPAM-Based Hydrogels. Homopolymerization of cyclic MDO monomer results in PCL formation through the RROP mechanism and in the present work this feature was utilized to engineer degradable polyNIPAM backbone. FIG. 1 illustrates the synthesis of a polyNIPAM-based hydrogel by photocopolymerization of NIPAM with MDO and PCLDMA degradable cross-linker that completely degrades to oligomeric units that can cleared from the body.

Copolymerization of NIPAM with MDO contributes ester linkages to the polymer backbone chains through incorporation of CL units and therefore leads to the formation of a degradable polyNIPAM backbone. Thus, by polymerization of NIPAM in presence of MDO and PCLDMA, one can obtain degradable sites throughout the polymer network (backbone and the crosslinks).

In order to demonstrate the impact of NIPAM copolymerization with MDO on the degradable properties of the polymer backbone, polyNIPAM-based hydrogels with the nondegradable cross-linker TEGDMA were synthesized. Nondegradable control, poly(NIPAM-TEGDMA), was prepared in absence of MDO; partially degradable poly(NIPAM-TEGDMA)-20 and poly(NIPAM-TEGDMA)-40 were synthesized in the presence of 20 and 40% mol/mol of MDO, respectively. The accelerated degradation of the hydrogels in 0.1N NaOH solution was evaluated by measuring weight loss (%) vs time and is summarized in FIG. 2.

FIG. 2 demonstrates that control, poly(NIPAM-TEGDMA) does not exhibit degradation while both poly(NIPAM-TEGDMA)-20 and poly(NIPAM-TEGDMA)-40 show significant weight loss of 48 and 65%, respectively, after 10 h of exposure to alkaline solution. The hydrogels were synthesized with nondegradable cross-linker and the modification was applied only in the polymer chain and therefore this experiment is a proof for incorporation of ester linkages into the polyNIPAM backbone by the presence of MDO during copolymerization. ¹H NMR of linear polyNIPAM-40 also confirms the presence of the CL units in the polyNIPAM backbone: ¹H NMR (CHCl₃) δ 1.14. (—CONCH(CH₃)₂), 1.26-1.93 (—CO₂CH₂CH₂CH₂CH₂—) and (—CH₂—CH(CONCH(CH₃)₂), 2.13 (—CH₂—CH(CONCH(CH₃)₂), 2.85 (—CH₂CO₂CH₂CH₂CH₂CH₂—), 3.54 (—CH₂CO₂CH₂CH₂—CH₂CH₂—), 4.03 (—CONCH(CH₃)₂).

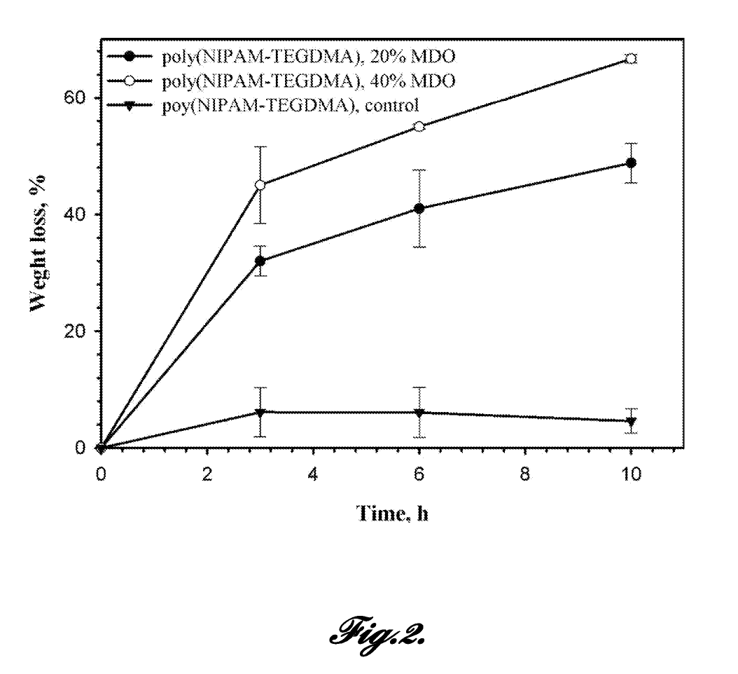

VPTT of fully degradable hydrogels, polyNIPAM-20 and polyNIPAM-40 (prepared in presence of 20 and 40% mol/mol MDO, respectively) and partially degradable polyNIPAM (prepared in presence of PCLDMA but in absence of MDO) was determined by DSC and defined as the onset temperature of the endotherms, as shown in FIG. 3.

Both polyNIPAM-20 and polyNIPAM-40 exhibit a VPTT around 30° C. compared to polyNIPAM homopolymer with a VPTT of 32.1° C. In general, VPTT of themosensitive polyNIPAM-based hydrogels is usually governed by the relative hydrophobicity of the bulk. As a result of the copolymerization of NIPAM with MDO, the hydrophobic/hydrophilic balance of the polyNIPAM hydrogel is changed due to incorporation of CL units into the polyNIPAM backbone. The presence of the hydrophobic CL fragments increases the hydrophobicity of the hydrogel network and therefore decreases the VPTT of fully degradable polyNIPAM-20 and polyNIPAM-40 compared to partially degradable polyNIPAM. The poly NIPAM-40 has been suggested to have more hydrophobic character then polyNIPAM-20 since it was prepared with a higher molar percent of MDO and consequently contains more CL fragments. This assumption can explain the slight difference in the VPTT values of polyNIPAM-20 and polyNIPAM-40 hydrogels: 30.2 and 30.6° C., respectively. The relatively large breadth of the endotherms, which is observed for polyNIPAM-20, polyNIPAM-40 and polyNIPAM samples, probably can be related to gradual, not sharp, deswelling of the hydrogels.

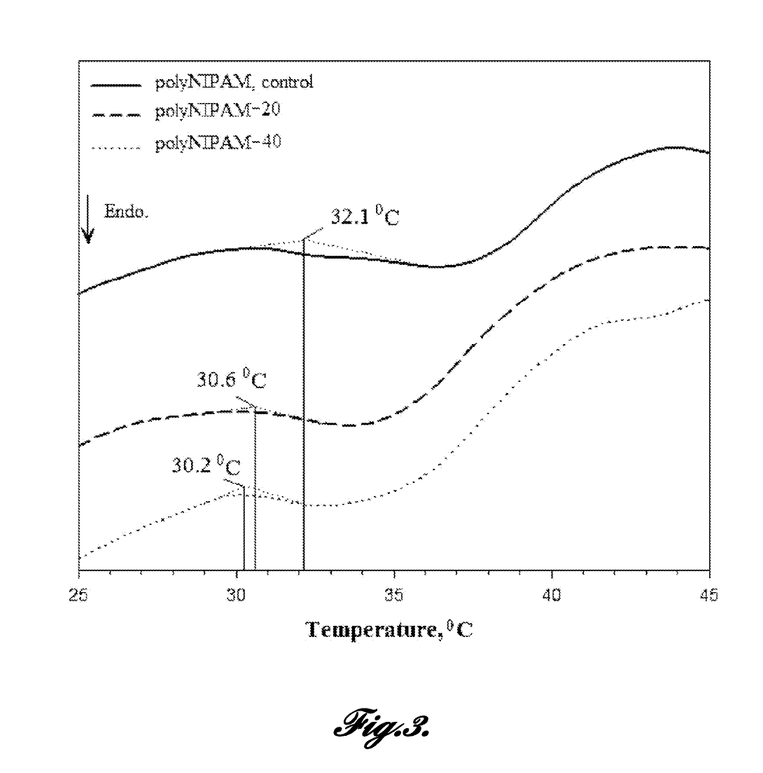

FIG. 4 shows the swelling of polyNIPAM-20, polyNIPAM-40 and polyNIPAM hydrogels at temperatures below and above the VPTT.

As seen in FIG. 4, polyNIPAM-20, polyNIPAM-40, and polyNIPAM hydrogels decrease in swelling as temperature increases, which is the same tendency in thermoresponsive behavior consistent with previously reported data regarding polyNIPAM-based materials. A significant decrease in swelling occurs at 37° C., the temperature above the VPTT, due to intensive dehydration of the polymer network.

FIG. 4 also demonstrates that at each temperature, the hydrogels exhibit slightly different swelling ability that depends on their copolymer composition. As a result of copolymerization with MDO, both polyNIPAM-20 and polyNIPAM-40 possess increased hydrophobic nature compared to polyNIPAM and therefore show lower swelling. The polyNIPAM-40 exhibits lower swelling at each temperature comparing to polyNIPAM-20, as shown in FIG. 4, which can be explained by the increased hydrophobicity.

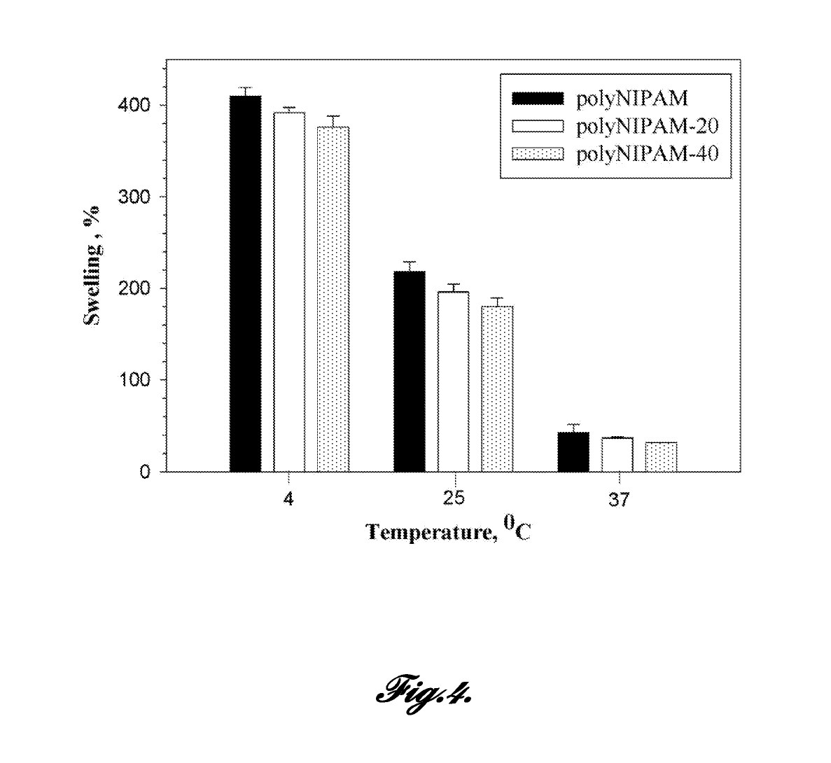

FIG. 5 shows polyNIPAM-40 hydrogel disks that were swollen in water at 4, 25, and 37° C. to visualize the difference in swelling at temperatures below and above the VPTT.

FIG. 5 demonstrates a decrease in the diameter of the swollen disks with increasing temperature (the initial diameter of the three dry disks before swelling was exactly the same). Visual inspection of the polyNIPAM-40-based hydrogel indicates a transparent appearance at the temperatures below VPTT and an opaque appearance at 37° C. due to dehydration of the bulk. Thus, the picture presented in FIG. 5 supports the results summarized in FIG. 4.

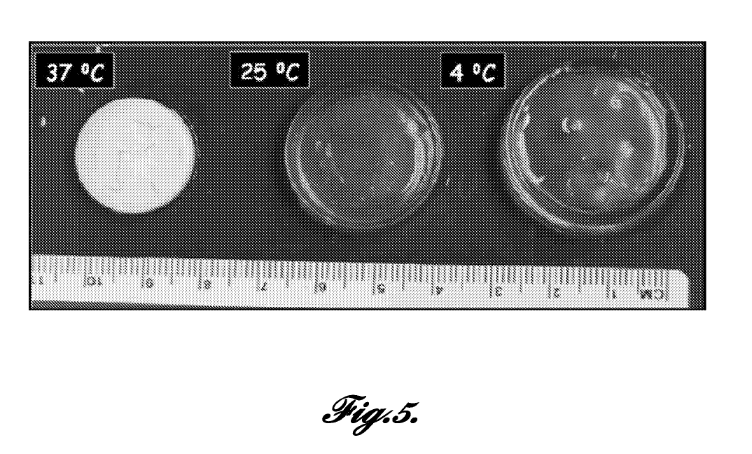

To explore the temperature-dependent viscoelastic properties of polyNIPAM-20 and polyNIPAM-40, the storage modulus, G′, was measured under oscillatory shear conditions. FIG. 6 demonstrates the storage modulus of fully degradable polyNIPAM-20 and polyNIPAM-40 hydrogels as a function of temperature.

Both polyNIPAM-20 and polyNIPAM-40 exhibit the typical rheological behavior of thermosensitive polyNIPAM-based materials. As shown in FIG. 6, the VPTT there are nonsignificant changes in values of the storage modulus with increase in temperature. At approximately 30° C., the temperature corresponding to the VPTT, there is an increase in G′, which becomes more dramatic with increasing temperature. This behavior indicates the deswelling for both hydrogels leading to the formation of more rigid materials.

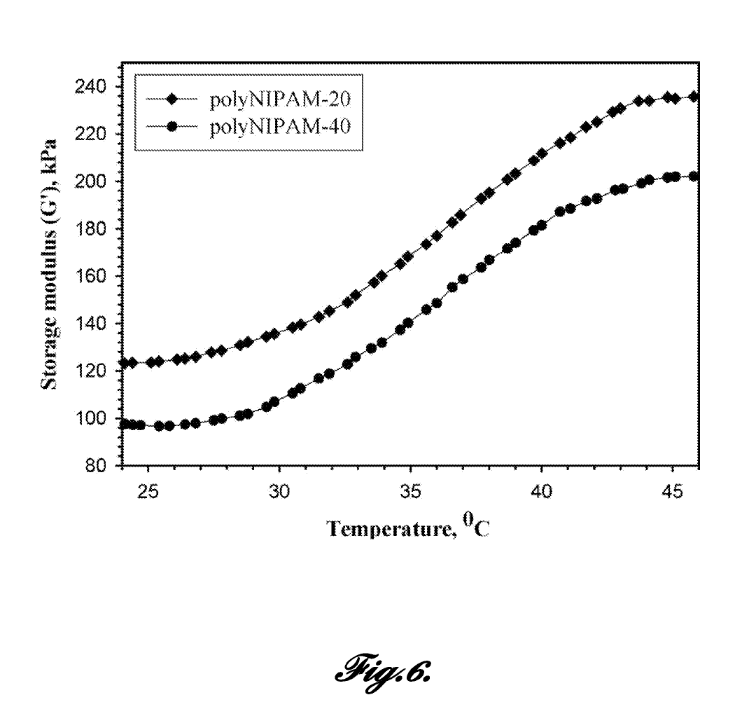

FIG. 7 demonstrates the degradation of polyNIPAM-20 and polyNIPAM-40 hydrogels at 25 and 37° C. that was monitored by measuring weight loss of the hydrogels in 0.007 N NaOH as a function of time. FIG. 7 shows that the degradation rate of both hydrogels at 37° C. is significantly slower than at 25° C. This behavior is reasonable, since at 25° C. the hydrogels are more swollen by alkaline solution and degradable sites are available and accessible for accelerated hydrolysis. On the other hand, at 37° C., the temperature above the VPTT, the hydrogels had expelled much water. This increases the hydrogels' bulk hydrophobicity and affects chain conformation by collapse due to dehydration. Now degradable sites are less accessible for hydrolysis and polyNIPAM-20 and polyNIPAM-40 probably degrade through surface erosion, which can explain the decreased degradation rate. FIG. 7 also demonstrates that at both temperatures, polyNIPAM-40 exhibits higher weight loss as compared to polyNIPAM-20. For example, after 24 h of degradation at 25° C. polyNIPAM-20 and polyNIPAM-40 lost 29 and 51% of the initial weight, respectively, and after 5 days of degradation at 37° C. polyNIPAM-20 and polyNIPAM-40 exhibit decreases of 6 and 18% of their initial weight, respectively. It can be assumed that polyNIPAM-40 has more degradable sites since it was prepared in the presence of a higher concentration of MDO and therefore degrades faster than polyNIPAM-20.

In this example, degradation of the polyNIPAM-20 and polyNIPAM-40 hydrogels was explored in an alkaline environment rather than at physiological pH in order to accelerate the degradation process of the PCL blocks. PCL is a well-known biodegradable polyester used in biomedical applications with degradation kinetics considerably slower than other aliphatic polyesters due to its hydrophobicity and crystallinity. PCL containing materials degrade in vitro (under physiological conditions) and in vivo on time scales of 3-6 month to 2 years, depending on composition, PCL molecular weight, etc. In the present work we structured our experiments to demonstrate that our new polymers can indeed degrade to soluble products. Since PCL polymers have often been shown to appropriately degrade upon in vivo implantation, these new polymers should behave similarly under in vitro accelerated conditions. When we focus this polymer system on a specific application with defined degradation time requirements, we will modify our material accordingly and explore degradation under the appropriate physiological conditions.

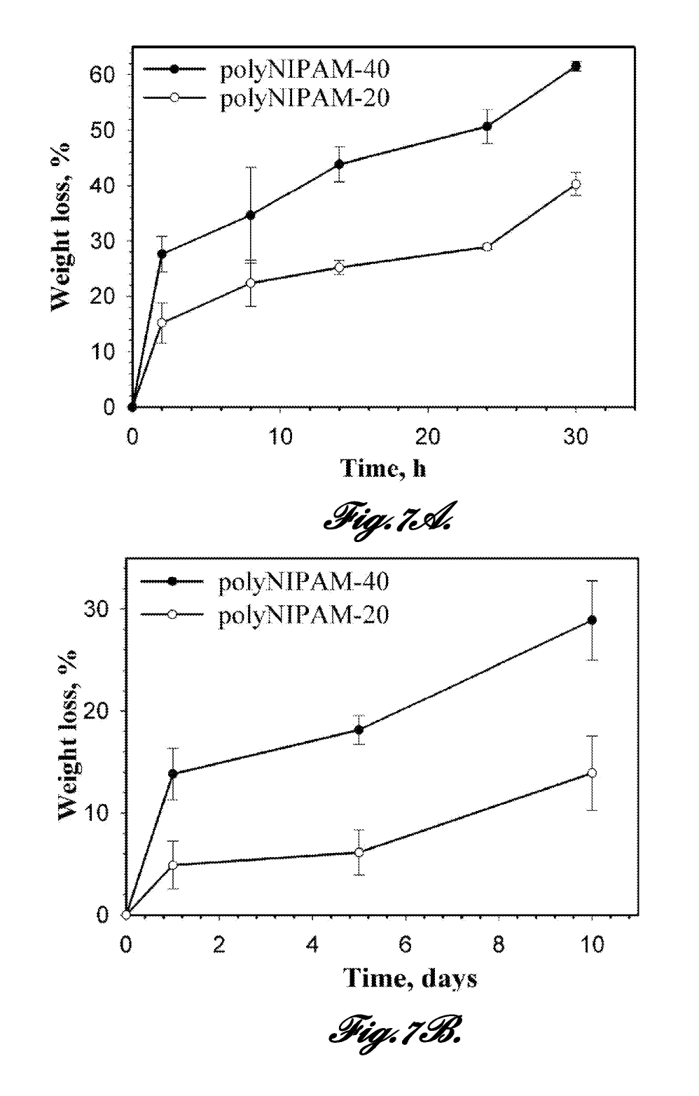

Fully Degradable PolyNIPAM Scaffolds. FIG. 8 illustrates the process for the sphere-templated fabrication of fully degradable, highly interconnected polyNIPAM-based scaffolds with thermally controllable pore size. This specific approach to sphere-templating was developed by Ratner et al. and is disclosed in U.S. Patent Application Publication No. 2008/0075752, the disclosure of which is incorporated herein by reference in its entirety. The method involves forming a cross-linked polymer scaffold around a template of sinter-fused, monodisperse-sized porogens and removing the template to produce a porous biomaterial.

In this study, monodispersed PMMA microspheres of the desired diameter are introduced into the mold, sintered to fuse particles, infiltrated with a monomer mixture of NIPAM, MDO, PCLDMA, and photoinitiator dissolved in DMSO, polymerized in situ to obtain a composite of polyNIPAM hydrogel and PMMA particles and finally exposed to solvent to dissolve the PMMA particles. This yields a fully degradable, porous highly interconnected scaffold. In summary, the pore size of the scaffold is defined by the initial diameter of PMMA particles and the interconnectivity is obtained through sintering for longer or shorter times.

This sphere-templating protocol differs from previous versions in the use of DMSO as the monomer solvent. Previous methods used water to dissolve monomers for poly(2-hydroxyethyl methacrylate) (polyHEMA) or fibrin scaffold fabrication. In the present example, water could not be used as a solvent because MDO is moisture sensitive and the PCL-based cross-linker is not soluble in water. Thus, we needed to develop a system based on an organic solvent that, on the one hand, led to a homogeneous solution of the monomers and reagents but, on the other hand, will not dissolve the PMMA templates during the infiltration. DMSO was found to meet these requirements and the sphere-templated procedure was adjusted to the present reagents composition.

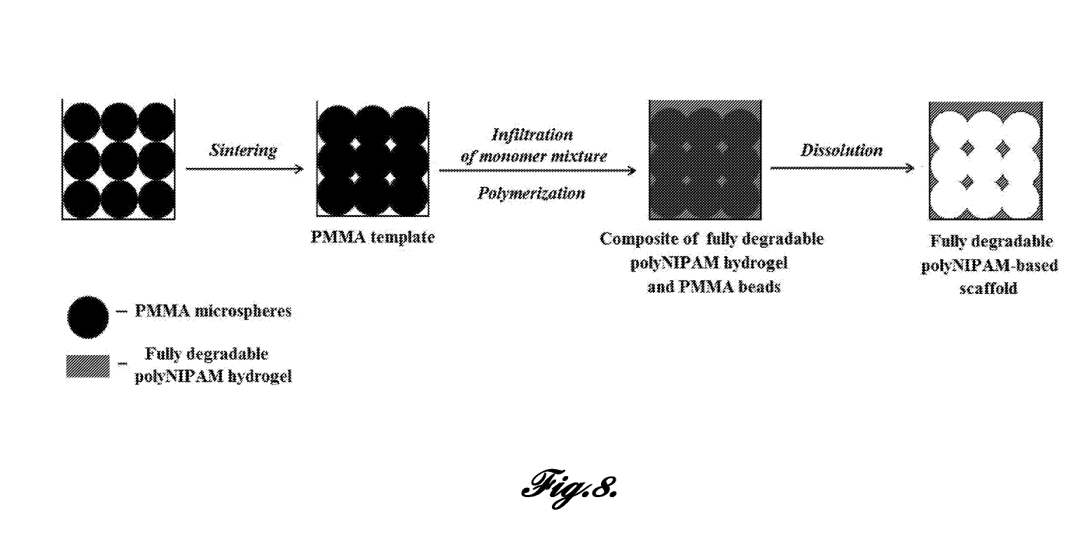

Fully degradable scaffolds based on the polyNIPAM-40 hydrogel with pore diameters of 36±2, 55±5, 90±8, and 204 (26 μm (at 25° C.) were fabricated by using templates composed of PMMA beads of 35±1, 49±8, 81±8, and 188±26 μm, respectively. FIG. 9 shows a representative SEM image of the polyNIPAM-40-based scaffold with a 55±5 μm pore diameter.

TABLE 2
Shrinkage at 37° C. in PolyNIPAM-40-
based Scaffolds with Different Pore Diameters

| | |
|---|---|
| pore diameter, μm | shrinkage, % |

| 25° C. | 37° C. | linear | volumetric |
|---|---|---|---|
| 36 ± 2 | 29 ± 1 | 20 | 48 |
| 55 ± 5 | 39 ± 3 | 29 | 64 |
| 90 ± 8 | 67 ± 4 | 25 | 59 |
| 204 ± 26 | 138 ± 19 | 32 | 69 |

The shrinkage study was performed as described in the Experimental Section.

The image demonstrates a monodispersed highly interconnected porous morphology that is typical for the scaffolds of all pore sizes.

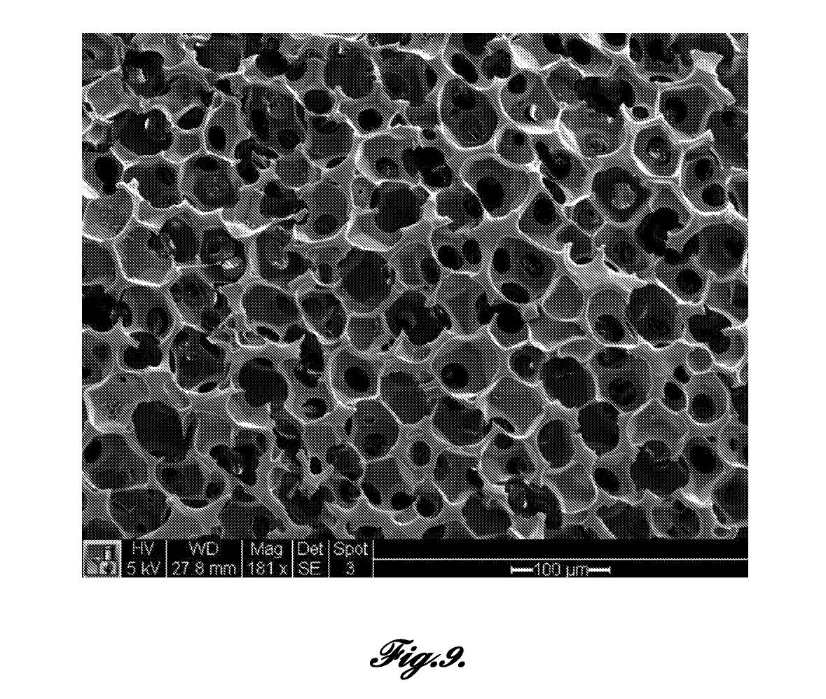

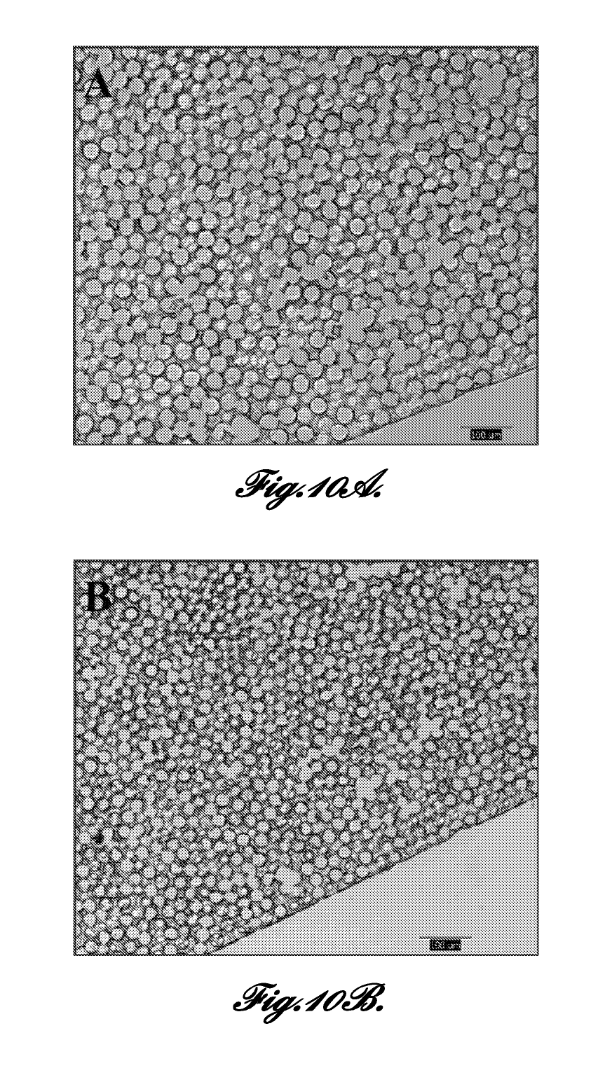

Our objective was to achieve a change in pore size of the polyNIPAM-40-based scaffolds going from RT to body temperature. FIG. 10 shows light microscope images of the scaffold's cross-section with pore diameter of 36±2 μm at 25 (A) that was reduced to 29±1 μm at 37° C. (B).

FIGS. 10A and 10B represents the same area of the section at the same magnification, and it can be qualitatively seen that at 37° C. (FIG. 10B) the pores shrank. The shrinkage can be attributed to volume phase transition since the temperature was raised above the hydrogel's VPTT.

Table 2 quantifies the percent shrinkage at 37° C. in polyNPAM-40-based scaffolds with different pore sizes.

Table 2 demonstrates the decrease in pore diameter at 37° C. for polyNIPAM-40-based scaffolds of all explored pore sizes. For example, the scaffolds with pore diameter of 36 and 90 μm exhibit linear shrinkage in a pore size of about 20 and 25%, respectively. Based on the aforementioned results one can assume that the shrinkage might affect not only the pore size but also the pore throat diameter (which is about 30% of the pore size for the sintering conditions used). This must be taken into consideration when planning scaffolding based on thermosensitive polyNIPAM because at body temperature the throat size should be large enough for efficient cell distribution throughout the scaffold. For example, at 25° C. the throat size of the scaffold with 36 μm diameter is about 11 μm but at body temperature it is probably will be reduced by approximately 20% and could be too small for cell penetration. Therefore, polyNIPAM-40-based scaffolds that have potential for tissue engineering should have a pore size starting at 55 μm at room temperature for sufficient throat size at body temperature.

The fact that the polyNIPAM-40-based scaffold can be engineered to have larger pores at room temperature than at 37° C. makes the scaffold attractive for cell loading and delivery. We have a special interest in scaffolds with pore diameters of 35-40 μm because in previous studies we found that monodisperse pores of approximately 35-40 μm are optimal for vascularized, relatively nonfibrotic healing. This was seen with both silicone elastomer and polyHEMA based scaffolds. Cells have been found to show an increased adhesion to the polyNIPAM-based materials at 37° C. due to protein adsorption to the more hydrophobic polymer. Thus, we hypothesized that due to the relatively large pore size and low cell-adhesiveness, the polyNIPAM-40-based scaffolds with pore diameter of 55 μm may efficiently be loaded with cells at RT and then at 37° C. the cells will be locked, both geometrically and with cell-polymer adhesive interactions, within the scaffold with optimal pore size of 39 μm. Thus, upon implantation, the cell-loaded spliere-templated scaffold could sustain the loaded cells through enhanced angiogenesis and decreased fibrosis, and, after providing mechanical and biochemical support, may degrade leaving behind regenerated, vascularized tissue. Results regarding cell loading will be presented later in the article.

An additional benefit of the poly-NIPAM scaffolds is a unique synthetic polymer composition allowing physicochemical properties to be tuned for optimal performance in tissue engineering. For example, degradation rate of the scaffold can be controlled by the molar ratio between NIPAM and MDO monomers, as well as by cross-linker nature and network density.

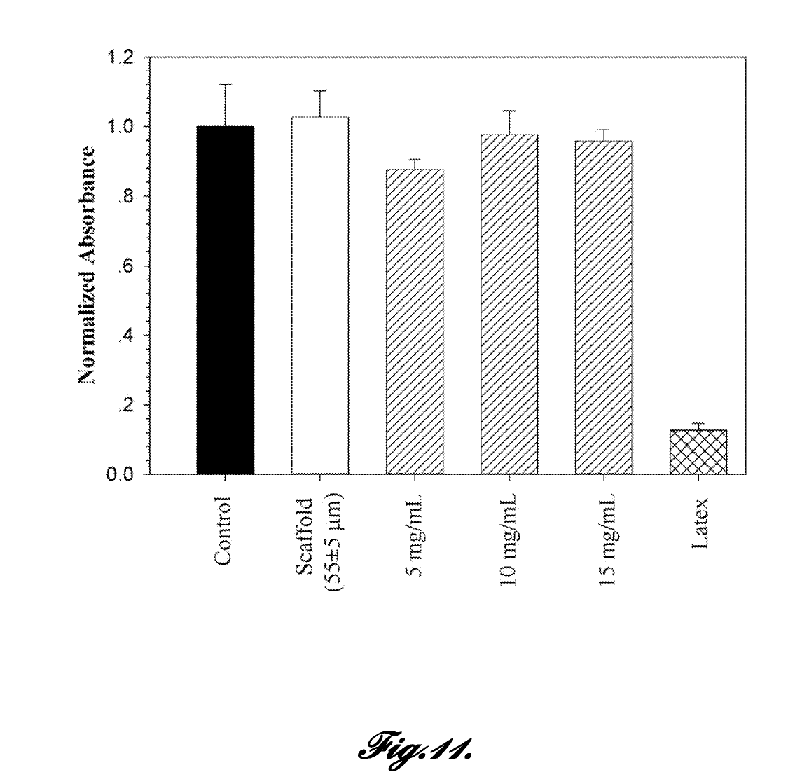

The in vitro cytotoxicity of the fully degradable scaffolds and their degradation products were evaluated by a colorimetric MTT test. MTT is a yellow tetrazolium salt that is reduced to form violet formazan only in living, metabolically active cell mitochondria and thus, the number of living cells can be spectrophotometrically quantitated. In the present study, the absorbance values of the samples were normalized to the negative control (TCPS). FIG. 11 shows representative results of the MTT test for the scaffold with 55±5 μm pore diameter and its degradation products compared to negative and positive (latex) controls.

From data presented in FIG. 11, it can be concluded that neither the scaffold itself nor its degradation products in the concentration range between 5-15 mg/mL are cytotoxic toward fibroblast cells. The same results were obtained for polyNIPAM-20 and polyNIPAM-40-based scaffolds of different pore sizes (data not shown).

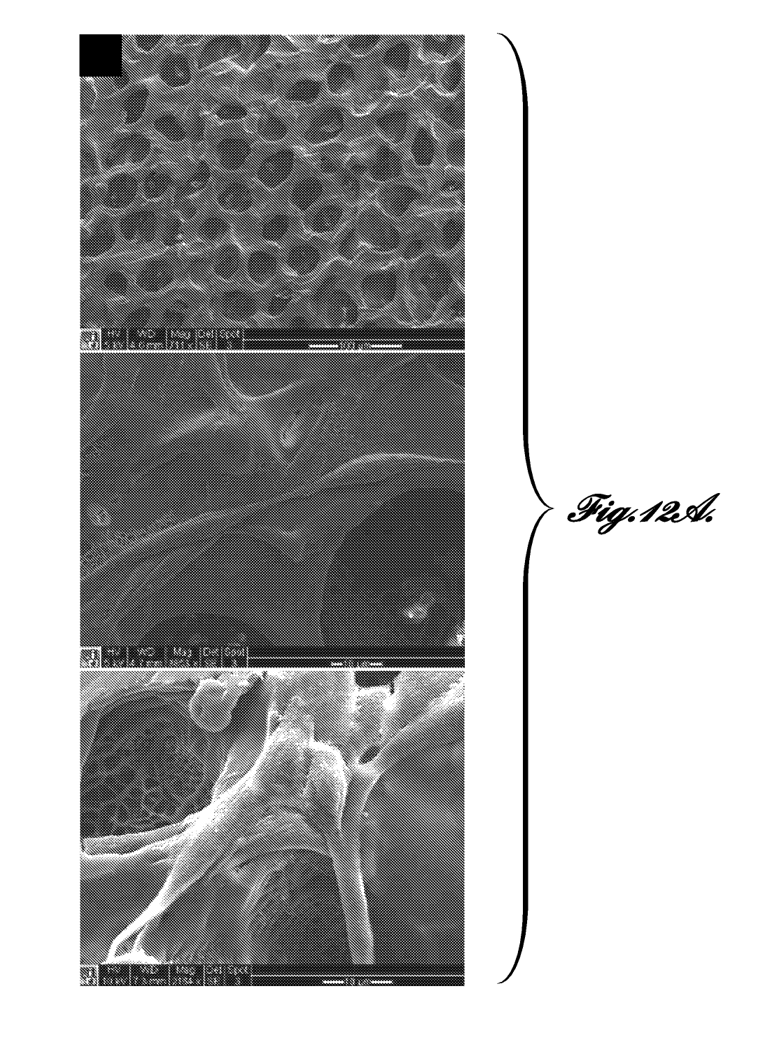

In order to explore the adhesion of the cells to the polyNIPAM-40-based scaffold, the 55±5 μm pore diameter was used as a surface for culturing fibroblast for 2 and 5 days. Representative SEM images of the scaffold's surface at different magnifications after 2 days of cell culture are seen in FIG. 12A. The cells are properly attached to the surface and show appropriate morphology. After 5 days of culture (FIG. 12B), the formation of a cell sheet on the surface of the scaffold as well as cell infiltration within the pores was observed. This behavior indicates the ability of the polyNIPAM-40-based scaffold to support cell attachment and growth and suggests cell compatibility with the material.

To demonstrate the potential of the polyNIPAM-40-based scaffold material for tissue engineering, the scaffold with a 55±5 μm pore diameter was loaded with a model system for many cell types, NIH3T3 cells. The scaffold with this particular diameter was chosen since its diameter decreases at 37° C. to 39 μm (Table 2). As was mentioned, this pore size was found to be optimal for vascularized, relatively nonfibrotic integration into tissue.

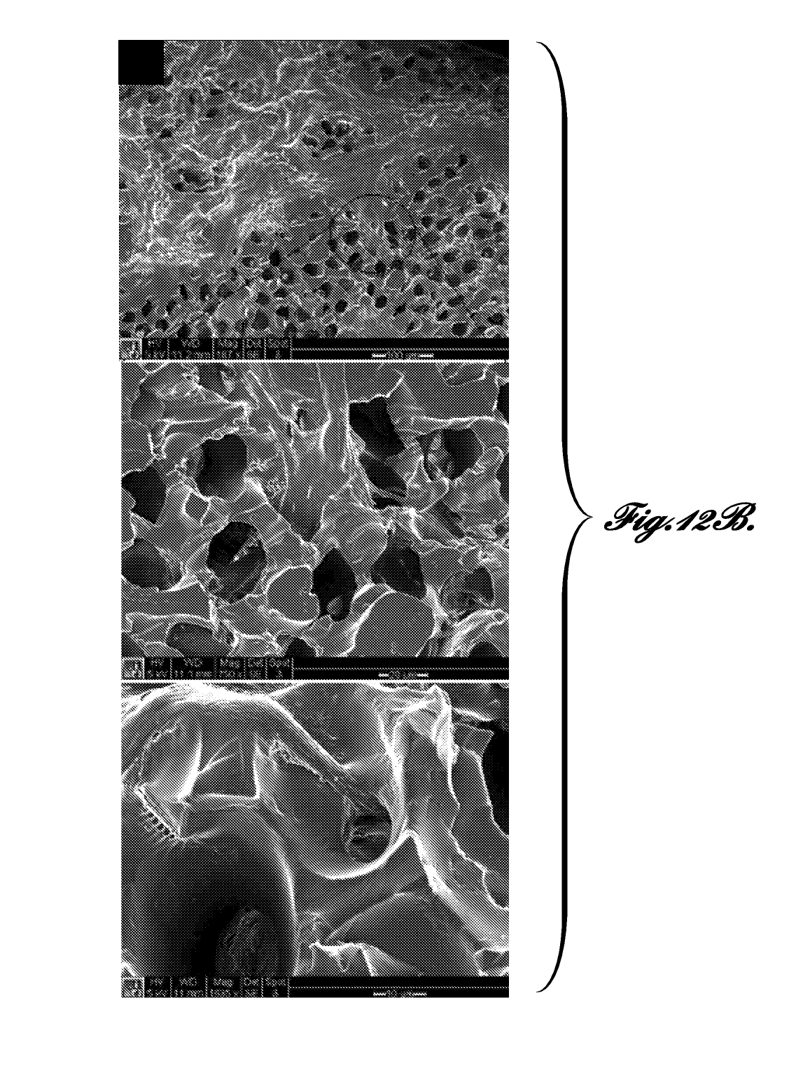

FIG. 13 shows relatively uniform cell distribution within the scaffold from the top to the bottom. The denser cell population on the one of the edges indicates the direction of the cell seeding. The magnified images show cell infiltration through the interconnecting throats and their appropriate elongated morphology.

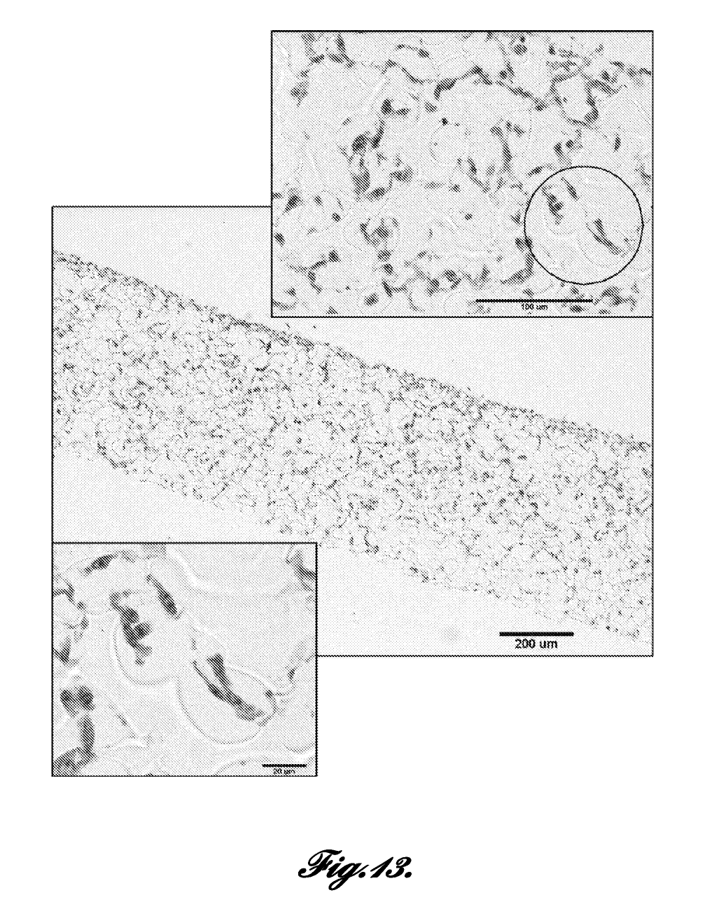

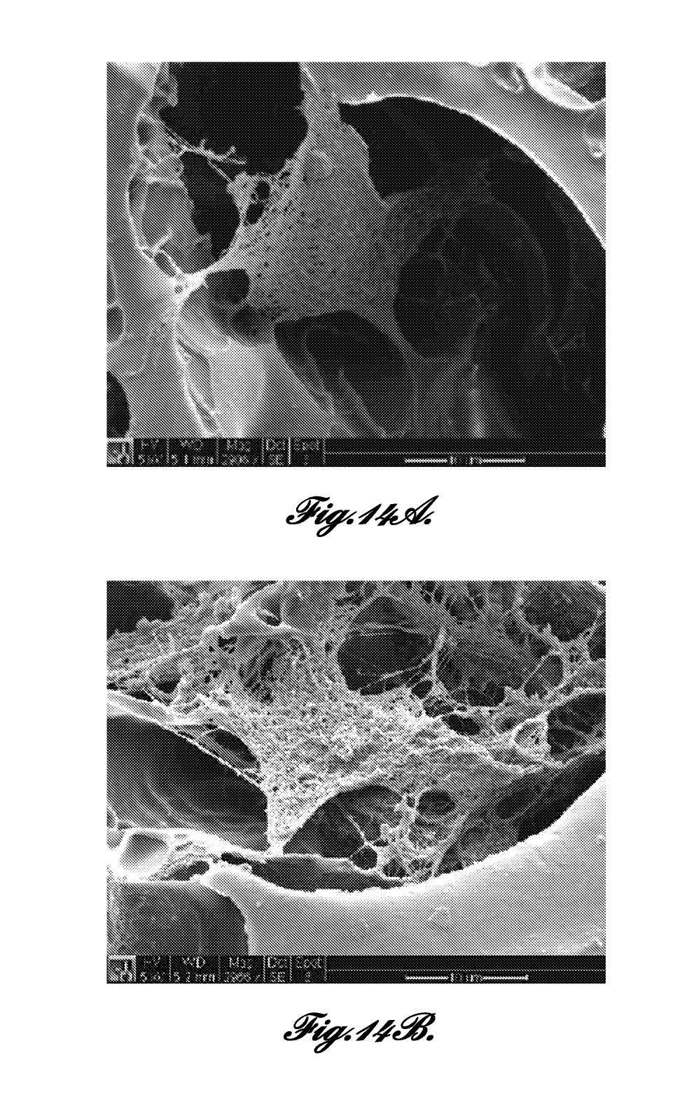

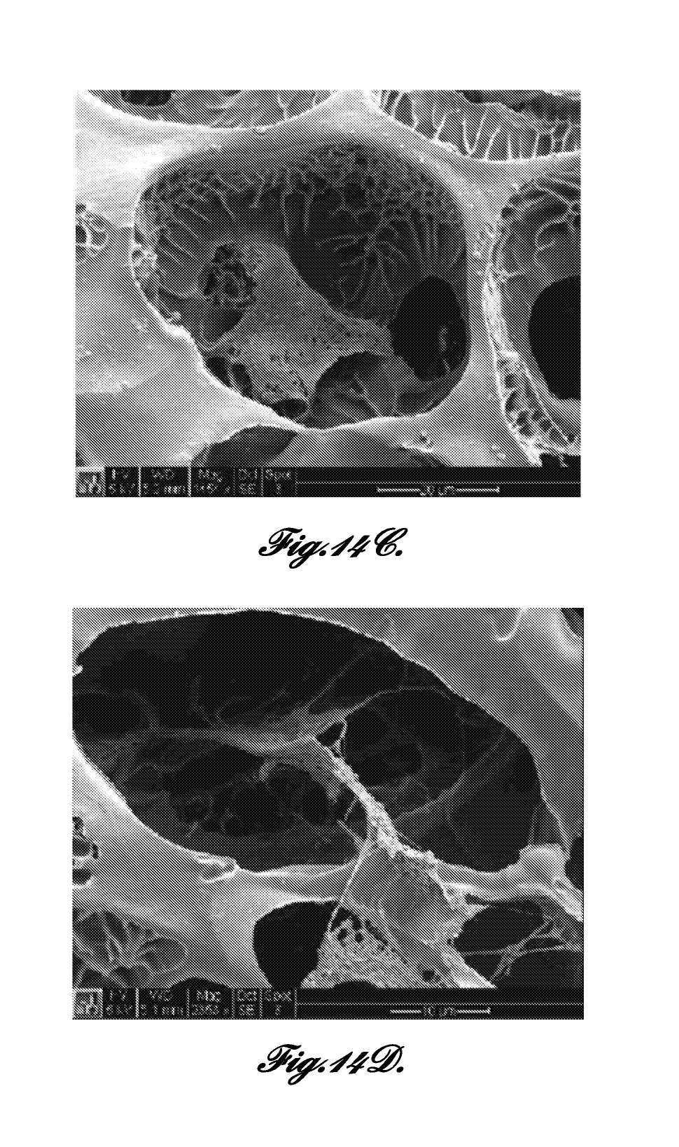

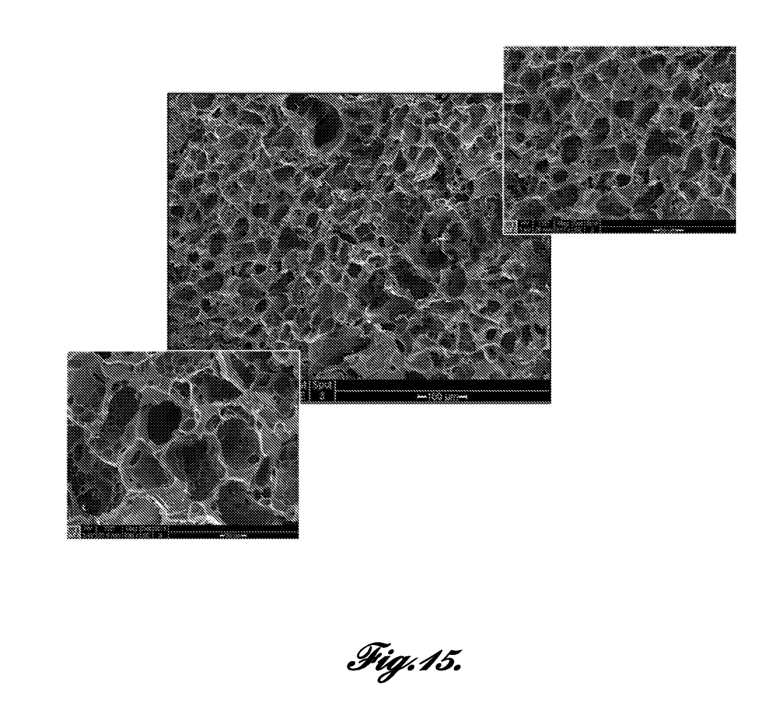

Representative SEM images of the cell-loaded polyNIPAM-40-based scaffold with a pore diameter of 55±5 μm (at room temperature) are shown in FIG. 14. The cross-section images demonstrate cell attachment and infiltration throughout the scaffold and support the histological data.

Thermosensitive scaffolds with two important criteria, full degradability and controlled porosity, were developed. First, fully degradable polyNIPAM-20 and polyNIPAM-40 hydrogels with ester linkages within both the backbone and the crosslinking sites were synthesized and characterized. Second, porous, highly interconnected polyNIPAM-40-based scaffolds with defined pore sizes were fabricated by using the sphere templating technique. The scaffold with a pore diameter of 55±5 μm was efficiently loaded with fibroblast cell at 25° C. The potential to use the scaffolds for tissue engineering was demonstrated by increasing the temperature to 37° C., thereby locking cells in the pro-angiogenic 39 μm pores. Future studies on the scaffolds will focus on loading the scaffold with 55±5 μm pore diameter with cells and following their proliferation in vitro, as well as implantation of the scaffold in an animal model and the exploration of its behavior in vivo. Due to controlled monodisperse pore diameter, highly interconnected morphology, fully degradable nature and thermoresponsive properties, this novel polyNIPAM-based scaffold is an attractive candidate for applications in tissue engineering.

### Example 2

### Hydrogel Scaffold Formed Using Salt Leaching

A fully biodegradable pNIPAM-based scaffold was also fabricated using a salt leaching/gas foaming technique. To define pore size of the scaffold, NaCl crystals were sifted prior the scaffold fabrication to provide relatively uniform crystals. Two different scaffolds were formed, one scaffold having crystals of 38-54 μm and another having crystals of 118-200 μm. For salt leaching/gas foaming technique NaCl crystals of the desired size range were mixed with 10% w/w of ammonium carbonate. To fabricate the scaffold, first NaCl/ammonium carbonate crystals were introduced into the mold and sonicated for optimal packing. Next, the template was infiltrated with a monomer mixture that contains NIPAM, MDO, PCLDMA and photoinitiator dissolved in DMSO. The monomeric mixture was then polymerized in situ to obtain a composite of pNIPAM hydrogel and NaCl/ammonium carbonate crystals. The last step is dissolution of the NaCl/ammonium carbonate in 50% aqueous citric acid and then water to obtain a desirable scaffold.

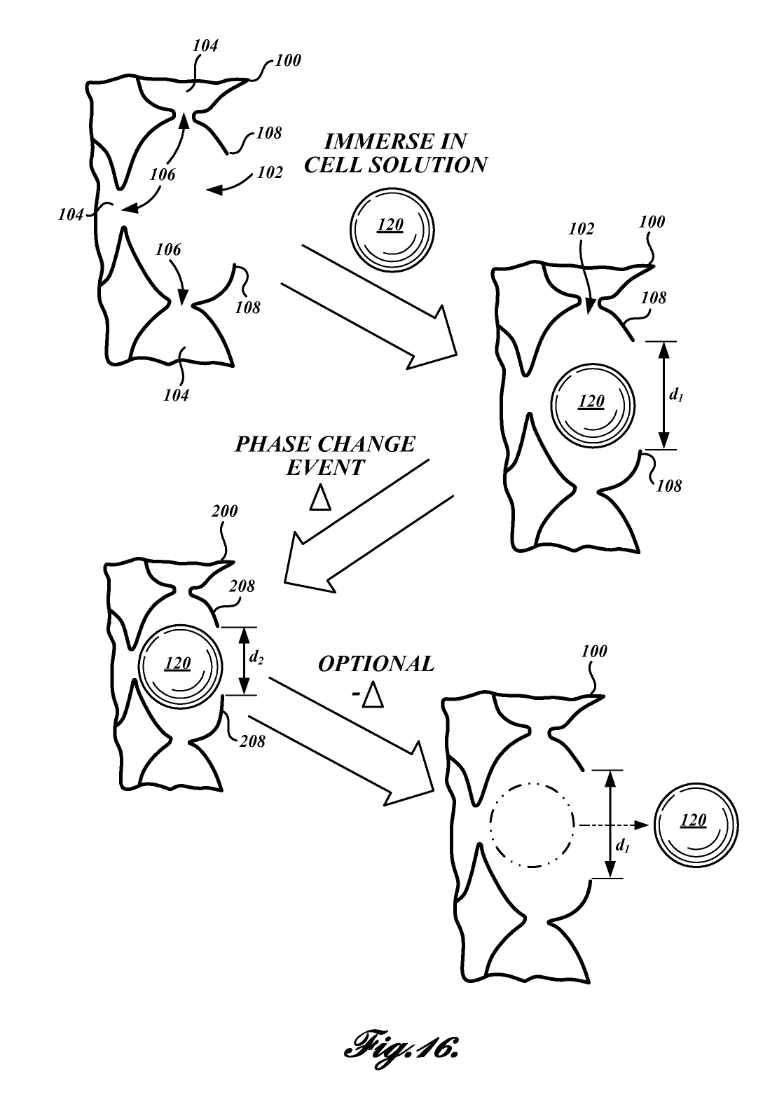

FIG. 15 is a composite of three SEM images of a pNIPAM-based scaffold with a 40±10 μm pore diameter that were fabricated using NaCl crystals of 38-54 μm.

While illustrative embodiments have been illustrated and described, it will be appreciated that various changes can be made therein without departing from the spirit and scope of the invention.

The embodiments of the invention in which an exclusive property or privilege is claimed are defined as follows:

1. A scaffold formed from a stimuli-responsive polymer, the scaffold having a plurality of interconnected pores that each has a volume that changes in relation to a phase transition event, wherein the phase transition event is initiated by an effective stimulus to the stimuli-responsive polymer.

2. The scaffold of claim 1, wherein the stimuli-responsive polymer is responsive to a stimulus selected from the group consisting of temperature, pH, electrical field, magnetic field, light, radiation forces, salt concentration, calcium concentration, and combinations thereof.

3. The scaffold of claim 1, wherein the stimuli-responsive polymer comprises polymers selected from the group consisting of poly (N-isopropyl acrylamide), poly methacrylic acid poly(ethylene oxide)-poly(propylene oxide)-poly(ethylene oxide), poly(ethylene glycol)/polyester copolymers, poly(vinylpyrrolidone)-based polymers, elastin peptides, and natural modified polymers, such as methylcellulose, chitosan, and xyloglucan, and combinations thereof.

4. The scaffold of claim 1, wherein the stimuli-responsive polymer is a hydrogel prior to the phase transition event.

5. The scaffold of claim 1, wherein the scaffold becomes less hydrophilic after the phase transition event.

6. The scaffold of claim 1, wherein the stimuli-responsive polymer comprises a backbone copolymer comprising a stimuli-responsive polymer portion and a biodegradable polymer portion.

7. The scaffold of claim 6, wherein the biodegradable portion is selected from the group consisting of oligo or polycaprolactone (PCL)-based segments, PCL-PEG-PCL, PLA-PEG-PLA, PLGA-PEG-PLGA, PCL-PEG-PPG-PCL, and combinations thereof.

8. The scaffold of claim 6, wherein the biodegradable portion comprise a moiety selected from the group consisting of an ester, an amide, a phosphazine, an anhydride, an orthoester, and a disulfide.

9. The scaffold of claim 6, wherein the biodegradable polymer portion will biodegrade in vivo to yield oligomeric units that can be excreted or otherwise biologically cleared from the body.

10. The scaffold of claim 9, wherein the oligomeric units comprise the stimuli-responsive polymer portions.

11. The scaffold of claim 1, wherein the stimuli-responsive polymer comprises a plurality of biodegradable crosslinking moieties that crosslink the stimuli-responsive polymer.

12. The scaffold of claim 11, wherein the biodegradable crosslinking moieties are selected from the group consisting of derivatives of poly(ε-caprolactone) (PCL), polylactic acid, dextran, chitosan, disulfide, amino-acid and polyaspartic acid.

13. The scaffold of claim 11, wherein the biodegradable crosslinking moieties comprise a moiety selected from the group consisting of an ester, an amide, a phosphazine, an anhydride, an orthoester, a polyhydroxyalkanoate, and a disulfide.

14. The scaffold of claim 11, wherein the biodegradable crosslinking moieties will biodegrade in vivo to yield oligomeric units that can be excreted.

15. The scaffold of claim 14, wherein the oligomeric units comprise the stimuli-responsive polymer.

16. The scaffold of claim 1, wherein the stimuli-responsive polymer is a temperature-responsive polymer and the phase transition event is initiated by changing the temperature of a solution in which the scaffold is immersed, wherein raising the temperature of the solution from a first temperature that is below the phase transition temperature to a second temperature that is above the volume phase transition temperature results in a shrinking of the volume of the pores of the scaffold.

17. The scaffold of claim 1, wherein the pores have a shape selected from the group consisting of circles and ovals.

18. The scaffold of claim 1, wherein the pores have a diameter of from 10 micrometers to 500 micrometers.

19. The scaffold of claim 1, wherein the scaffold is angiogenic.

20. A method for forming the scaffold of claim 1, comprising
(a) providing a template comprising a plurality of packed particles in a vessel, said packed particles being packed in an arrangement such that voids in between the particles can be infiltrated by a liquid poured into the vessel;
(b) filling the voids in between the particles with a solution comprising a monomer for the stimuli-responsive polymer and a polymerization initiator;
(c) polymerizing the monomer for the stimuli-responsive polymer using the polymerization initiator to provide the stimuli-responsive polymer as a matrix surrounding the packed particle template, thereby providing a particle-scaffold composite; and
(d) dissolving the packed particles from the particle-scaffold composite to provide the scaffold.

21. The method of claim 20, wherein the solution further comprises a monomer with a crosslinking moiety and wherein the step of polymerizing the monomer for the stimuli-responsive polymer using the polymerization initiator further comprises polymerizing the monomer with the crosslinking moiety to provide a crosslinked stimuli-responsive polymer as a matrix surrounding the packed particle template.

22. The method of claim 20, wherein the packed particles are packed in a configuration selected from the group consisting of simple cubic, body-centered, face-centered cubic, irregular, and hexagonal close-packed.

23. The method of claim 20, wherein dissolving the packed particles comprises submerging the particle-scaffold composite in a solvent for the particles.

24. The method of claim 20 further comprising a step of heating the template to a temperature sufficient to sinter the particles together prior to filling the voids in between the particles.

25. The method of claim 20, wherein the particles are a material selected from the group consisting of poly(methyl methacrylate), polystyrene, sucrose, sodium chloride, and poly(ethyl methacrylate).

26. The method of claim 20, wherein the particles are monodisperse spheres having a size of from 10 micrometers to 500 micrometers.

27. The method of claim 20, wherein the particles are a salt-leaching/gas foaming mixture comprising sodium chloride and ammonium carbonate and wherein dissolving the particles comprises submerging in aqueous acid and then water.

28. The method of claim 20, wherein the polymerization initiator is a photoinitiator and wherein the polymerizing step of the method comprises exposing the solution to electromagnetic radiation having a wavelength and intensity sufficient to activate the photoinitiator.

29. A method for growing cells using a scaffold of claim 1, wherein the scaffold has a large pore state and a small pore state depending on whether a volume phase transition event has occurred, the method comprising the steps of:
(a) contacting a suspension of cells with the scaffold in the large pore state such that the cells infiltrate the large pores of the scaffold;
(b) applying an effective stimulus to the scaffold so as to transition the scaffold to the small pore state, thereby trapping the cells in the pores;
(c) placing the scaffold containing trapped cells in a location where cell growth is desired;
(d) culturing the cells within the scaffold, which provides mechanical and biochemical support for the cells; and
(e) biodegrading the scaffold to provide space for aggregated cells or newly formed tissue.

30. The method of claim 29, wherein the cells are selected from the group consisting of endothelial cells, fibroblasts, macrophages, smooth muscle cells, mesenchymal stem cells, hematopoetic stem cells, embryonic stem cells, hepatocytes, cardiomyocytes, neurons, keratinocytes, osteoblasts, chondrocytes, and combinations thereof.

31. The method of claim 29, wherein the location is selected from the group consisting of in vitro and in vivo.

32. The method of claim 29, wherein culturing the cells within the scaffold comprises enhanced angiogenesis and decreased fibrosis, and wherein the cell proliferation results in vascularized tissue.
